(19) United States
(12) Patent Application Publication
(10) Pub. No.: US 2025/0253034 A1
(43) Pub. Date: Aug. 7, 2025

(54) MAPPING ENVIRONMENT WITH SENSORY PROSTHESES

(71) Applicant: COCHLEAR LIMITED, Macquarie University, NSW (AU)

(72) Inventors: Alexander VON BRASCH, Macquarie University (AU); Stephen FUNG, Macquarie University (AU)

(21) Appl. No.: 18/856,167

(22) PCT Filed: Apr. 12, 2023
(86) PCT No.: PCT/IB2023/053753

§ 371 Date: Oct. 11, 2024

Related U.S. Application Data
(60) Provisional application No. 63/330,028, filed on Apr. 12, 2022.

(51) Int. Cl. G16H 30/40; G16H 20/40; H04R 25/00

(52) U.S. Cl. CPC G16H 30/40; G16H 20/40; H04R 25/507; H04R 25/554; H04R 2460/01

(57) ABSTRACT

A system including a light sensor and/or a sonic sensor and/or a radio wave sensor and a processor configured to receive input based on light and/or sonic frequency reflection and/or radio wave reflection captured by the respective sensor and analyze the received input to develop a data usable to control a sensory prosthesis based on the received input.

### CROSS-REFERENCE TO RELATED APPLICATIONS

This application claims priority to U.S. Provisional Application No. 63/330,028, entitled MAPPING ENVIRONMENT WITH SENSORY PROSTHESES, filed on Apr. 12, 2022, naming Alexander VON BRASCH as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a system comprising a light sensor and/or a sonic sensor and/or a radio wave sensor and a processor configured to receive input based on light and/or sonic frequency reflection and/or radio wave reflection captured by the respective sensor and analyze the received input to develop a data usable to control a sensory prosthesis based on the received input.

In an exemplary embodiment, there is a non-transitory computer readable medium having recorded thereon, a computer program for executing at least a portion of a method, the computer program including code for analyzing first data based on data captured by a light sensor and/or sonic sensor and/or radio wave sensor code for quantitatively and/or qualitatively identifying a device performance impacting feature of an environment in which the light sensor and/or sonic sensor and/or radio wave sensor is located based on the analysis of the first data.

In an exemplary embodiment, there is a method, comprising obtaining first data based on captured light and/or sonic energy and/or radio frequency energy captured during a first temporal period utilizing an electronic device during the first temporal period and at least one of identifying an action to improve efficacy of a prosthesis based on the obtained first data or developing a sensory habilitation and/or rehabilitation tool and/or engaging in a sensory habilitation and/or rehabilitation program based on the obtained first data.

A system comprising a LiDAR capable optical sensor suite and a processor in signal communication with the optical sensor suite and configured to receive input from the optical sensor suite indicative of light captured by the optical sensor suite and analyze the received input to develop a data usable to control a sensory prosthesis based on the received input.

### BRIEF DESCRIPTION OF THE DRAWINGS

Embodiments are described below with reference to the attached drawings, in which:

FIG. 1 is a perspective view of an exemplary hearing prosthesis;

FIG. 2 presents a functional block diagram of an exemplary cochlear implant;

FIG. 3A and FIGS. 3B and 3C present exemplary systems of communication between devices;

FIG. 4 presents an exemplary retinal prosthesis;

FIG. 5 presents an exemplary vestibular implant;

FIG. 6 presents exemplary system diagram;

FIG. 7 presents an exemplary algorithm for an exemplary method;

FIG. 8 presents an exemplary prosthesis;

FIGS. 9 and 10 present an exemplary model based on LiDAR data;

FIGS. 11-13 present exemplary scenarios applicable to teaching exemplary embodiments;

FIGS. 14-16 and 18 present exemplary algorithms for exemplary methods; and

FIG. 17 presents a functional diagram.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to determine the viability of various types of prostheses, such as, for example, a vestibular implant and/or a retinal implant, with respect to a particular human being. And with regard to the latter, the techniques presented herein are also described with reference by way of background to another illustrative medical device, namely a retinal implant. The techniques presented herein are also applicable to the technology of vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

Also, embodiments are directed to other types of hearing prostheses, such as middle ear implants, bone conduction devices (active transcutaneous, passive transcutaneous, percutaneous), and conventional hearing aids. Thus, embodiments are directed to devices that include implantable portions and embodiments that do not include implantable portions.

Any reference to one of the above-noted sensory prostheses corresponds to an alternate disclosure using one of the other above-noted sensory prostheses unless otherwise noted, providing that the art enables such.

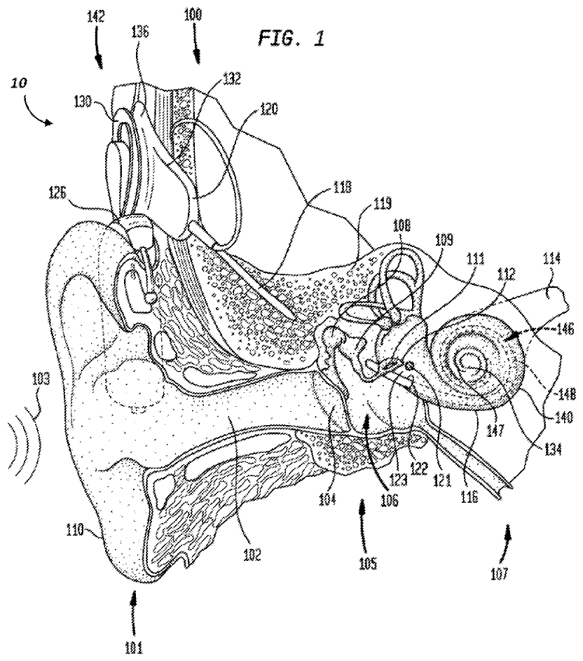

FIG. 1 is a perspective view of a cochlear implant, referred to as cochlear implant 100, implanted in a recipient, to which some embodiments detailed herein and/or variations thereof are applicable. Particularly, as will be detailed below, there are aspects of a cochlear implant that are utilized with respect to a vestibular implant, and thus there is utility in describing features of the cochlear implant for purposes of understanding a vestibular implant. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). Again, it is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

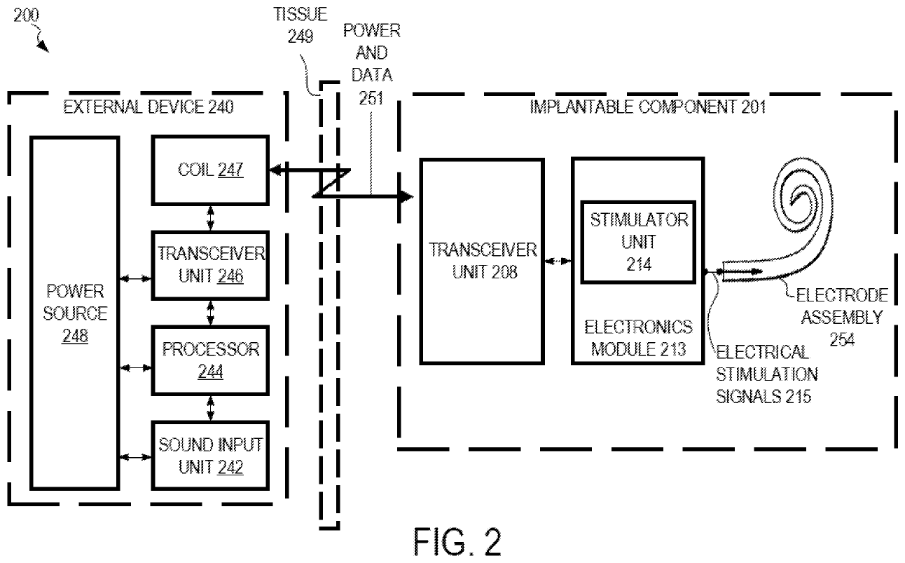

FIG. 2 is a functional block diagram of a cochlear implant system 200 to which the teaching herein can be applicable. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power, because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

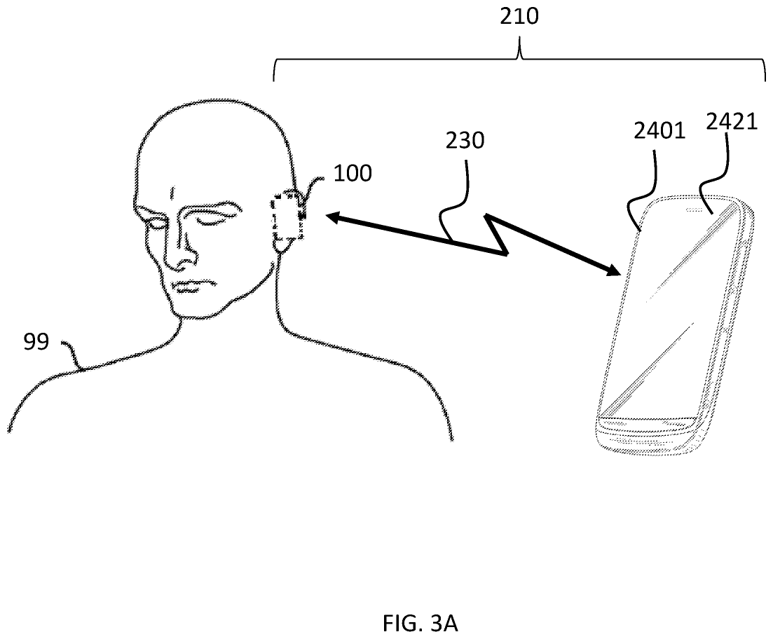

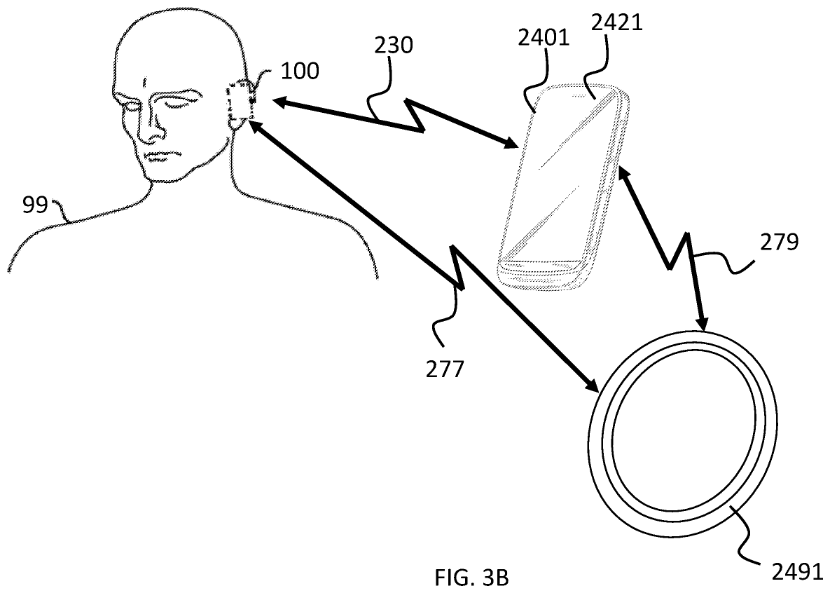

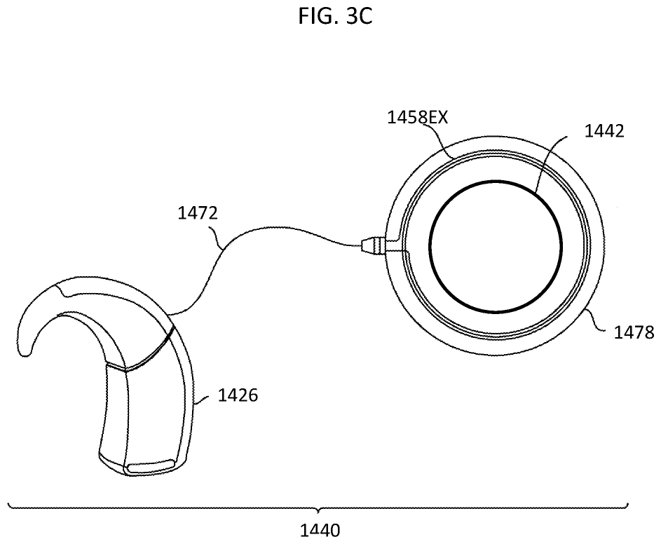

FIG. 3A depicts an exemplary system 210 according to an exemplary embodiment, including hearing prosthesis 100, which, in an exemplary embodiment, corresponds to cochlear implant 100 detailed above, and a portable body carried device (e.g., a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).)

In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Jan. 10, 2021. In an exemplary embodiment, the device 2401 corresponds to a Samsung Galaxy Gear™ Gear 2, as is available in the United States of America for commercial purchase as of Jan. 10, 2021. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 3B depicts an exemplary quasi-functional schematic depicting communication between an external communication system 2491 (e.g., a telecoil, or Bluetooth transceiver), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)). It is noted that unless otherwise noted, the embodiment of FIG. 3B is applicable to any body worn medical device/implanted device disclosed herein in some embodiments.

FIG. 3C depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

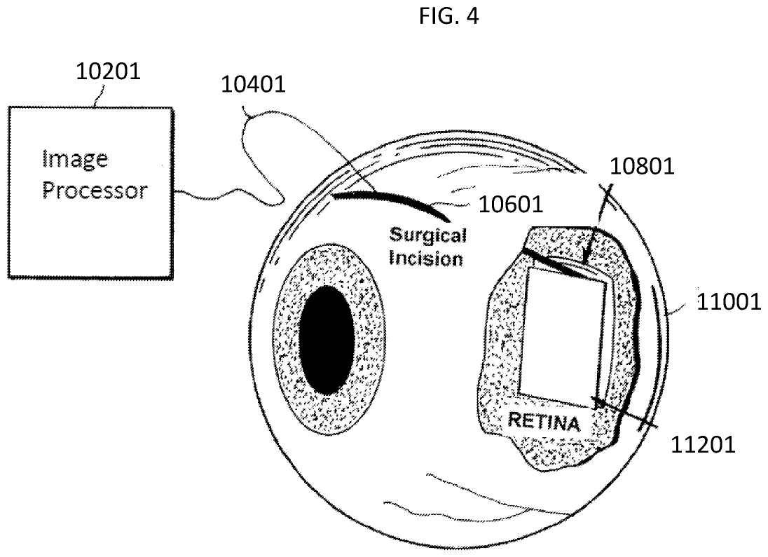

FIG. 4 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

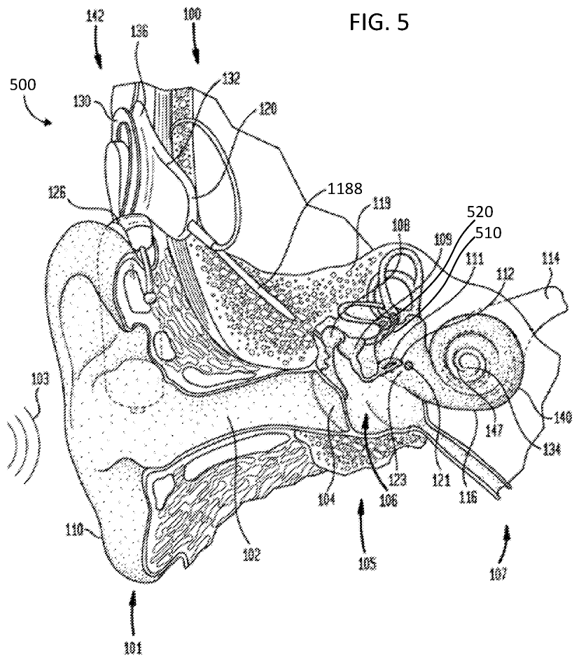

FIG. 5 depicts an exemplary vestibular implant 500 according to one example. Some specific features are described utilizing the above-noted cochlear implant of FIG. 1 in the context of a vestibular implant. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 5 represents a partially implantable vestibular implant, embodiments can include a totally implantable vestibular implant, such as, where, for example, the motion sensors are located in the implantable portion, in a manner analogous to a cochlear implant.

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 5. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

Embodiments can include sensory prostheses that use some form of beamforming to improve, for example, hearing performance in some situations. Some embodiments utilize an adaptive beamforming. This can be used to enhance the signal coming from specific directions (such as speech from the front), and/or suppress the sound coming from other directions (such as noise from the rear or sides), which is often unwanted or otherwise can obscure the desired source/noise (such as from the front, which can be a person speaking to the recipient of the prostheses). In some embodiments, this is achieved by analyzing the signal as received from the microphones, for devices that have multiple microphones.

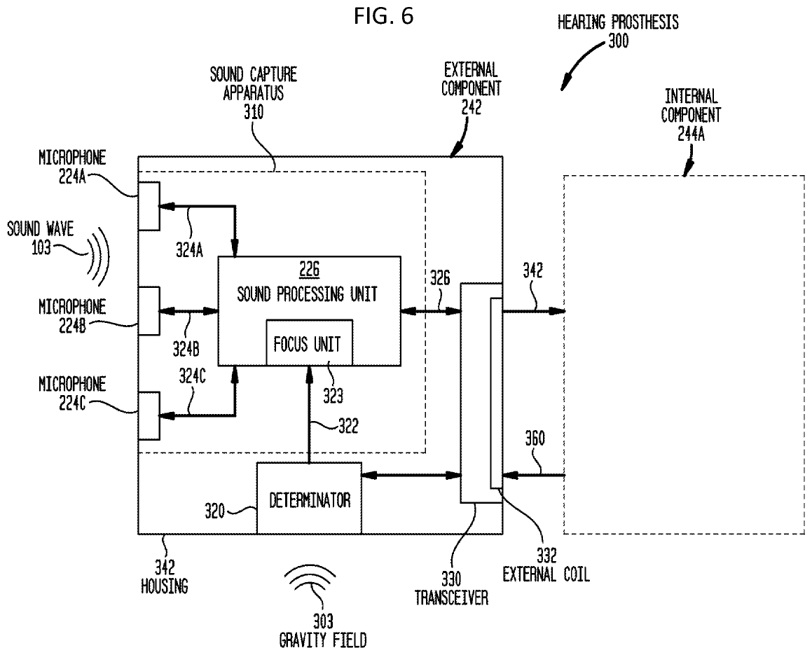

FIG. 6 depicts a functional block diagram of a hearing prosthesis 300 according to an exemplary embodiment that uses beamforming. In an exemplary embodiment, the hearing prosthesis 300 may correspond to the cochlear implant detailed above, with external component 242 corresponding to the external component detailed above, which can be the external component for any of the above-noted devices, and internal component 244A corresponding to the internal component detailed above, which can be the internal component/implanted component according to any of the devices detailed above (if such is the case-again, some embodiments do not include an implanted component, and thus the implanted component and the related features of the external component may not be present in such embodiments).

In the exemplary embodiment, the external component 242 includes microphones 224A, 224B and 224C that are in signal communication with sound processing unit 226 via signal routes 324A, 324B and 324C. Collectively, these components form a sound capture apparatus 310 The sound capture apparatus may also include a focus unit 323 that is part of the sound processing unit 226, but may also be a separate component from the sound processing unit 226. The sound processing unit 226 is in signal communication with transceiver 330, which includes a coil 332 (corresponding to the external coil referenced above with respect to FIGS. 2A and 2B). Sound processing unit 226 outputs signals indicative of processed captured sound via signal route 326 to transceiver 330, which transmits via external coil 332 an electromagnetic signal 342 to the internal component 244A. External component 242 also includes a determinator 320, which can be a processor or a computer chip programed to determine the orientation of the external device and/or the sound capture apparatus and, in an exemplary embodiment, is sensitive to a gravity field 303. The sound capture apparatus 310 and the determinator 320 can collectively form a sound capture system, although in some embodiments, the determinator is not present (reliance can be placed on the recipient to correctly place the external component in a utilitarian orientation). Also, in an exemplary embodiment, the sound processing unit 226 may be located in the internal component 244A of the hearing prosthesis 300.

In the embodiment, the microphones are arrayed on the front face of the housing 342 at about 120 degree intervals (e.g., at the 12 o'clock, 4 o'clock and 8 o'clock position-housing 342 can be an off the ear sound processor and can be at least generally circular, but note that two or three microphones can be arrayed on the BTE device for example, and similar principles can be used for beamforming for such an embodiment). However, in other embodiments, the microphones may be arrayed about the side of the housing 342 in a different manner. Microphones on the side and the front may be used in combination. Any spatial arrangement of three or more microphones may be used in some embodiments of the present invention if such spatial arrangement will permit such embodiments to be practiced. The external coil 332 of transceiver 330, the sound processing unit 226 and the microphones 224A, 224B and 224C (or fewer microphones) are packaged in a single unit within or otherwise on a housing 342. (In an exemplary embodiment, as noted above, the sound processing unit 226 may be located elsewhere.) Such a device is sometimes referred to as a coil sound processor, a coil microphone and/or an off the ear sound processor, etc., because the external coil that is used to communicate with the internal component 244A is housed within or otherwise supported by the same components that house or support the sound processor unit and/or the microphones. This is in contrast to an external component of a hearing prosthesis in which the microphone(s) and/or sound processor unit are housed in or otherwise located on a so-called behind-the-ear (BTE) unit that hooks around the ear and is in signal communication with an external coil remote from the housing of the BTE unit (which can have the beamforming features as well. In this regard, the external component 242, according to an embodiment of the present invention, is a device that is attachable to the head of a recipient without contacting the recipient's ear and/or otherwise being significantly supported by the recipient's ear or other appendage of the recipient. In an embodiment, the external component 242 is attachable to the head of the recipient such that a face of the external component 242 abuts the skin of the recipient and there is no contact and/or minimal contact with skin of the recipient by the sides of the external component 242.

FIG. 6 shows back-telemetry 360 from the internal component 244A which may exist in some embodiments.

The embodiment of FIG. 6 can enable focusing capability/directional capability may be achieved via beamforming, where the beamforming is achieved via signal processing to achieve spatial selectivity of the sound capture apparatus. The directional capability/focusing capability of the sound apparatus 310/sound capture system has utility in embodiments where it can be correlated to a specific area relative to the recipient. A sound capture apparatus configured to focus on an area, such as an area relative to the external component 242, permits the sound capture apparatus/sound capture system to be “focused” in a given direction so that sound originating or otherwise traveling from that direction is weighted relative to other sounds, thereby permitting the recipient to hear more of that sound than other sounds. Such a feature has utility in that users of hearing prostheses often seek to hear words spoken to them to the exclusion of other words spoken to others, and thus the sound capture apparatus 310 can be focused to better capture such spoken sounds. Specifically, because a user typically faces the direction from which the spoken words originate or otherwise travel from, an embodiment of the present invention permits the sound capture apparatus to focus in a specific direction relative to the external component 242 to better capture sound from that specific direction.

As noted above, the external component 242 is configured to adjust a focus of the sound capture apparatus 310. Some exemplary embodiments of such a configuration will now be described. It is noted herein that a sub-component described as having a given capability also means that a component including that sub-component has that capability. In an exemplary embodiment, referring to FIG. 6, the external component 242 includes a focus unit 323 (which can be a processor or a chip “programmed” or having access to programming to execute focusing for beamforming) that is configured to adjust the direction of focus of the sound capture apparatus 310. As depicted in FIG. 6, the focus unit 323 is part of sound processing unit 226. In some embodiments, the focus unit 323 may be a separate component from the sound processing unit 226 that is in signal communication therewith via a communication line. In an exemplary embodiment, the adjustment made by the focus unit 323 is made to adjust the focus (i.e., directionality) of the sound capture apparatus 310 such that the sound capture apparatus 310 focuses on the area in front of the recipient. Specifically, in an exemplary embodiment, the focus unit 323 is configured to adjust the focus of the sound capture apparatus 310 to focus on an area of interest. Such adjustment will typically focus on the area in front of the recipient during normal use of the external component 242. Focusing can be executed by weighting the signals from the various microphones relative to one another, or by other techniques of beamforming.

Embodiments include utilizing maps of a local environment in which a recipient of a sensory prosthesis is located. The maps can be created using, by way of example, LiDAR (light detection and ranging), or an ultrasonic arrangement, or a radar arrangement, and can be combined with artificial intelligence or otherwise machine learning to enhance the hearing perception of recipients in various environments, as will be further described below. It is briefly noted that in the interests of textual economy, reference may be made to one of the technologies but not the other. Any such reference corresponds to a disclosure of the alternate technology having those attributes/being used according to such unless otherwise noted providing that the art enables such.

LiDAR is capable of detecting and determining the distances of objects present in the environment. LiDAR can be used to generate high-resolution digital maps for navigation and improve accuracy for modelling that involves spatial distancing. The data gives the platform and/or model more useful and accurate information about the user's indoor and outdoor environments. LiDAR technology can be used for accurate determination of wind speed for example. This can have utilitarian value herein for beamforming (where the beamforming is skewed relative to the direct line of sight to the origin of the sound to account for wind, for example, and embodiments include using LiDAR to beamform based in part on windspeed). This can have utilitarian value for wind whistle accommodation/noise cancellation, for example, where the wind speed can be used to trigger/set a threshold for the noise cancellation of the hearing prosthesis.

LiDAR can be harnessed using smart phones that have such capability, or can be implemented in a dedicated handset, or can be integrated into the hearing prosthesis for example. An embodiment can use the iPhone 12™. Embodiments can use the LiDAR scanners in these smart phones or smart devices or another device to perform accurate 3D scanning of an environment, or at least develop limited ranging to objects in an environment.

Some embodiments can thus include a system that includes a sub-system that includes a remote sensor(s) which has the LiDAR capability to scan the objects in the environment. For instance, a smartphone that is equipped with the LiDAR cameras to support the real time scanning of objects in the environment, map out distances and/or placement and represent geometric objects as a set of finite elements. But again, this can be integrated into the sensory prosthesis, or another device.

The system can include a sub-system, which can be part of the smart phone, such as hand held computing device 2401, or any hand-held computing device, or part of a personal computer, etc., that includes a module collecting the data of geometric objects in a given environment and parsing them for further input to a model which can be running in the cloud and/or app(s), that is able to generate a replicant sound field and/or a replicant light field representing the input to the user/listener/viewer.

The system can include a sub-system that includes a processing element that, based on the determined sound field of the recipient, communicates suggested settings changes, such as for the beamformer, to the recipient to improve the hearing performance and/or vision performance in the environment in which the recipient is located. The system can further include a sub-system that includes a sound processor and/or light processor that can then use this additional input/information to enhance its directional processing, in addition to the analysis this sub-system can also operate based on the received signal from the sound capture device of the hearing prosthesis and/or the light capture device of the vision prosthesis.

Moreover, some embodiments can include a database/library, or at least have access to such that is able to be used by an AI system to learn the environments and corresponding device settings for a specific user, and can be trained or otherwise accessed to provide more accurate operation relative to that which would otherwise be the case.

Embodiments are often described in terms of a hearing prosthesis. However, embodiments of most of the teachings herein can be applicable to a vision prosthesis or a balance prosthesis. Briefly, the teachings below have an emphasis on the concept of utilizing technology to develop a map or a model of an environment in which a recipient is located or will be located. By way of example and not by way of limitation, this map can have utilitarian value with respect to determining ranges and/or distances or otherwise angles of various objects in the environment to the recipient, or, more accurately, to a receptor portion of the prostheses, such as a microphone with respect to hearing prostheses. And with respect to this embodiment, there can be utilitarian value with respect to ascertaining the range to an object so that the prostheses can better set or adjust gain. For example, the more distant the source of the sound, the more the gain might be increased relative to a closer source of the sound. Accordingly, embodiments include utilizing the LiDAR data or the like to ascertain or at least extrapolate distances and automatically adjust one or more control settings of the hearing prosthesis. With respect to a vision prosthesis, a spatial model can be utilized for enhancement purposes of certain objects that will be the basis of visual stimulation for the recipient. Further, obtaining distance data and/or orientation data can be utilized to skew or otherwise emphasize certain portions of the field-of-view over others to give a better perception of depth for example.

And with respect to balance prostheses, for example, estimating or determining orientations of objects, including for example the slope of land upon which a recipient is standing or walking, can have utilitarian value in for example adjusting/controlling the balance prosthesis to have the recipient lean more forward or to one side more than the other, for example. For example, if the recipient is walking on a surface that has a slope of 20° downward from the right side of the recipient to the left side of the recipient, the LiDAR data can be used to determine the presence and/or value of the slope, and the direction thereof, and the vestibular implant could be controlled to provide stimulation to the recipient so that the recipient will lean towards the right.

Some embodiments can utilize the LiDAR data to identify objects within an environment or even to ascertain and otherwise classify a given environment. Some embodiments include a hearing prosthesis that can classify an environment based on the sound that is captured. This is often called sound classification. Some embodiments include a hearing prosthesis that can classify an environment based on the location where the recipient is present, which location can be ascertained utilizing cell phone triangulation and/or global positioning systems. Embodiments according to the teachings detailed herein can also use or instead use the LiDAR data to classify the environment. Some embodiments include a system that has a sophisticated image recognition routine or algorithm that can analyze the LiDAR data to estimate or otherwise identify the type of environment or even a specific environment. With regard to the latter, the system can remember various locations where the recipient was previously located, and conclude, based on the LiDAR data, that the recipient is located in one of these locations. The system can then classify the environment or otherwise the location, and adjust or control the prosthesis accordingly (or recommend that such adjustment be done). For example, if the classification is that the recipient is in a highly reverberant sound area, for example, reverberant sound processing can be automatically executed.

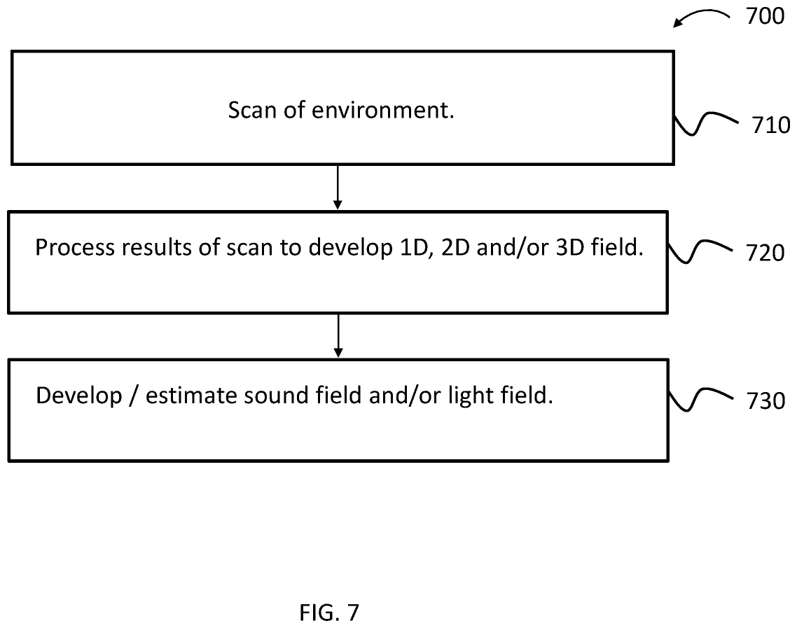

FIG. 7 presents an exemplary flowchart for an exemplary method, method 700, that includes method action 710. Method action 710 includes the action of scanning an environment in which the scanner of some exemplary embodiments of the teachings detailed herein is located. In an exemplary embodiment, this is executed in the environment in which the sensory prosthesis is utilized. But in some embodiments, this is not necessarily the case. In some embodiments, the scans of the environment are executed before the hearing prostheses is utilized in a given method, or otherwise with temporal separation between the utilization of the hearing prosthesis. In other embodiments, the scans are taken contemporaneously with the use of the hearing prosthesis. The various permutations of use will be described in greater detail below.

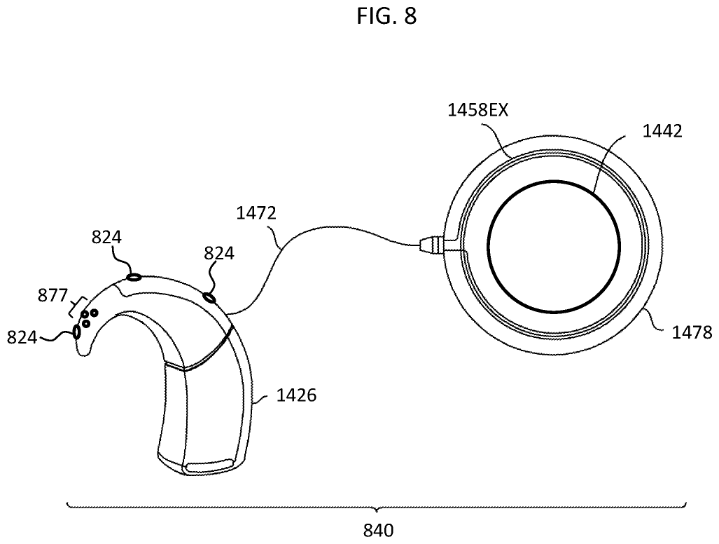

In an exemplary embodiment of method action 710, In some embodiments, the sensors that are utilized to execute method action 710 are part of a remote device separate from the hearing prostheses, such as being a part of the handheld device 2401 detailed above, or part of a laptop computer for example. In some embodiments, the sensors utilized to scan the environment are part of the sensory prosthesis. In this regard, FIG. 8 shows an exemplary external component of a hearing prosthesis 840. The external component includes a behind-the-ear device 1426, which includes three microphones 824 positioned as shown by way of example. These microphones can be utilized for beamforming or otherwise directionality of sound capture according to the teachings detailed herein. Behind-the-ear device 1426 further includes light sensors 877 as shown. These light sensors 877 can correspond to the light capture devices of a LiDAR system or any other system that can have utilitarian value with respect to enabling the teachings detailed herein. Indeed, by way of example, element 877 can correspond to a radar arrangement and/or a sonar arrangement such as, for example, an arrangement that utilizes ultrasonic waves that are generated by a transducer on the BTE device or on another device, the reflections off of objects being captured by transducers on the BTE device or on another device. Note further laser ranging can be used (an active system that uses light for example).

In this regard, while many embodiments are directed towards passive devices that collect ambient radiation, other embodiments can utilize more active devices such as the aforementioned sonar arrangements. Devices that rely on imaging can be devices that emit energy outside the visible spectrum that create a fluorescence or the like. Further, devices that utilize passive collection techniques can utilize the infrared spectrum. Any device, system, and/or method that can enable the teachings detailed herein can be utilized in at least some exemplary embodiments. Here, LiDAR has been chosen as but one example. And note that any disclosure herein of LiDAR includes a disclosure of the accompanying utilitarian software usable therewith which is available as of Jan. 10, 2022, in the United States, the United Kingdom, the Federal Republic of Germany, the Republic of France, The People's Republic of China, Australia, and/or New Zealand, etc. Of course, any disclosure herein of LiDAR corresponds to a disclosure of an alternate technology, such as radar or sonar, etc., that can enable the teachings detailed herein providing that the art enables such.

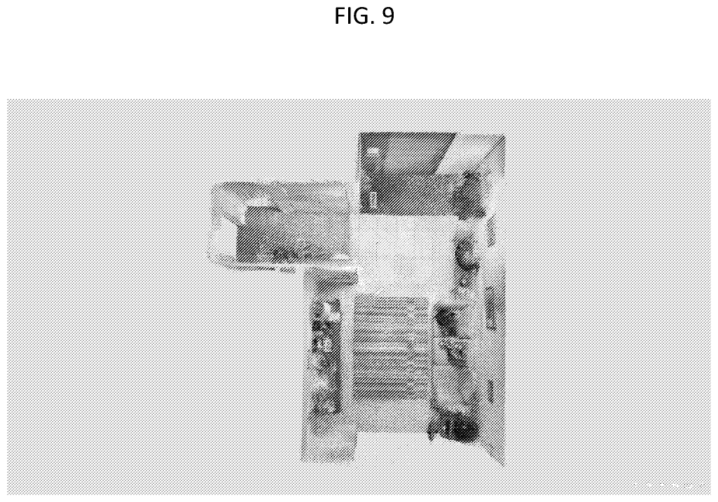

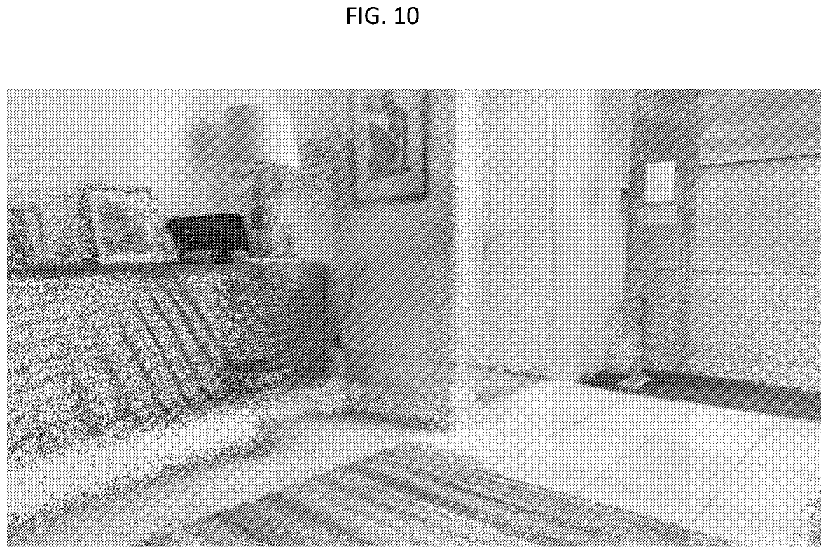

Method 700 further includes method action 720, which includes the action of processing the results of the scan of method action 710 to develop a 1D, 2D and/or 3D field. With respect to the latter, FIGS. 9 and 10 show depict exemplary results of processing the results of the scan of method action 710. FIG. 9 presents a three-dimensional top view of rooms of an apartment in which a recipient of the hearing prostheses inhabits, while FIG. 10 depicts a view of a room in which a recipient is sitting of the aforementioned apartment from the perspective of the recipient's BTE device while being worn on the right ear of the recipient.

Returning back to the flowchart of method 700, method 700 further includes method action 730, which includes the action of developing and/or estimating the sound field and/or a light field of the environment of method action 710. Here, where the embodiment under discussion is a hearing prosthesis or otherwise the teachings associated with the embodiment under discussion are to be utilized with a hearing prosthesis, the method action 730 entails developing a sound field. The developed sound field can take many forms. In one simple embodiment, the sound field can entail simply distances from the light capture sensors and/or the microphone(s) of the hearing prosthesis. In another embodiment, the sound field can entail distances and/or angles to those objects from the sensors and/or the microphones of the hearing prosthesis. Of course, in some other embodiments, the sound field can be a more developed sound field.

In an exemplary embodiment, method action 710 is an action that includes having LiDAR scanning objects and/or people in the room, and method action 720 includes using the LiDAR software to develop a high-resolution digital 3D representations of areas in the scanning environment, and this can be done statically and/or dynamically. For instance, the 3D representation can clearly indicate the relative distance and/or spatial orientation of the recipient (or of the LiDAR sensors and/or the microphone of the recipient's hearing prosthesis to his/her friend(s) and/or family, and/or co-workers, etc., sitting around a table. Upon using the LiDAR software to develop the relative spatial spacing of objects/people in the environment, the system can, in some embodiments, provide more accurate guidance to control and steer the beamforming directionality of the hearing prosthesis to focus on the source of signals of interest for better capturing the speech signals while reliably ignoring signals from other directions.

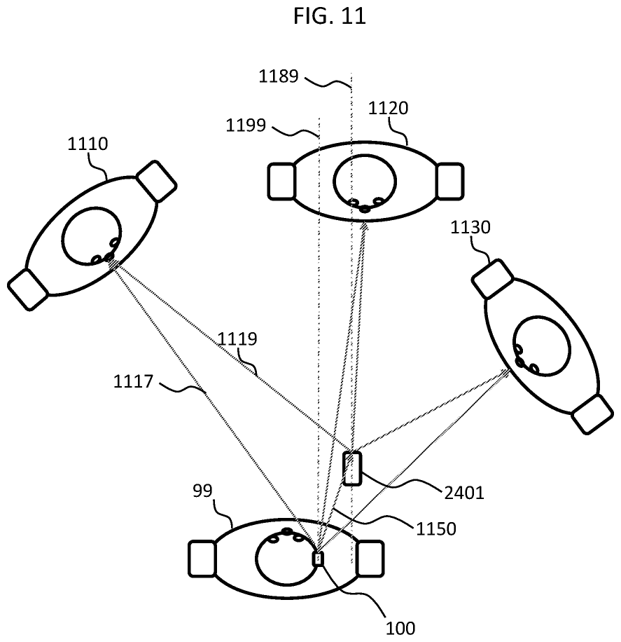

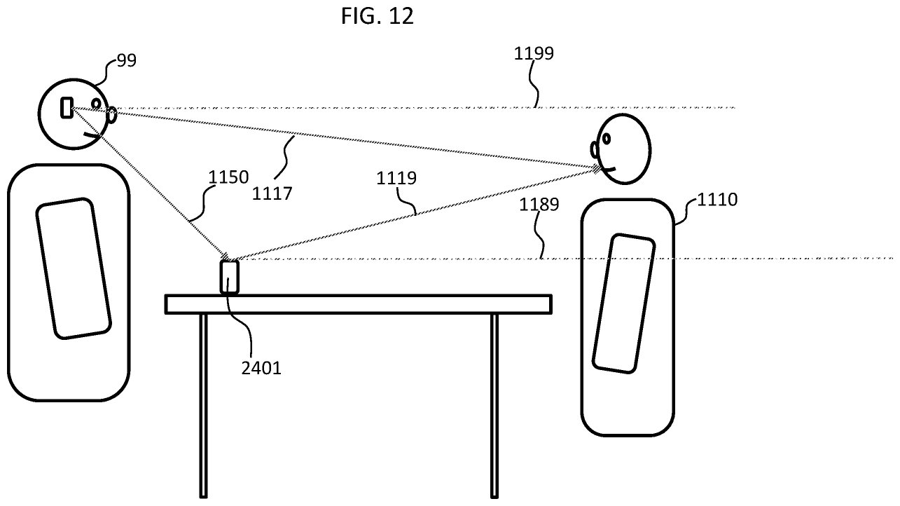

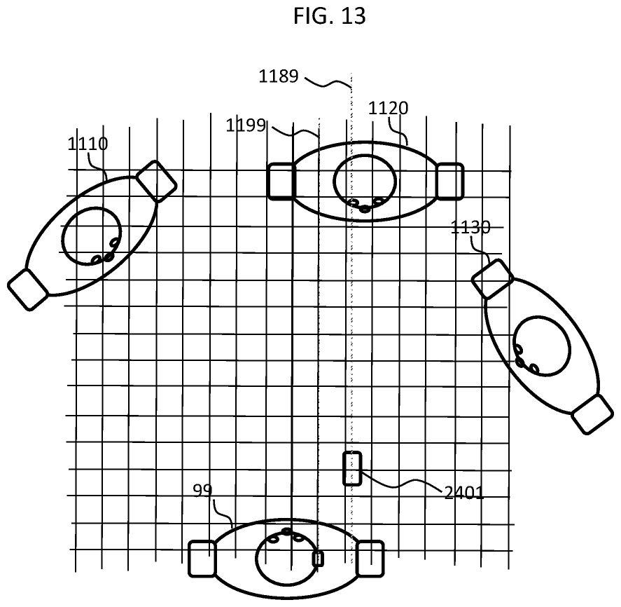

The LiDAR system can be utilized to develop a data set that can be utilized to implement beamforming. FIGS. 11 and 12 present an exemplary spherical coordinate system that can be developed utilizing the LiDAR system that can be centered off of the LiDAR device 2410 and/or the prostheses 100. In an exemplary scenario, the LiDAR system scans the area in front of the recipient 99 and obtains images of three people centered around the table (not shown), persons 1110, 1120, and 1130 in an exemplary embodiment, the LiDAR system can be sufficiently sophisticated so as to identify humans relative to other objects in a room, the center of masses of the humans, the heads of the humans, and/or even the mouths of the humans. The idea being that any beamforming could have utilitarian value with respect to focusing in on the mouths of a given speaker. In any event, shown in FIG. 11 and FIG. 12 are vectors directed towards the mouth of person 1110, and the center of masses of the remaining two people. As shown, vector 1119 extends from the LiDAR device 2401 and vector 1117 extends from the hearing prostheses external component 100 (again, the LiDAR device can be incorporated into the hearing prostheses). Also shown is vector 1150, which vector extends from the hearing prosthesis 100 to the LiDAR device 2401. Some of the aforementioned vectors can also be seen in FIG. 12. Also shown in the figures are axes 1199 and 1189. These axes can be respectively stable axes associated with the external component of the hearing prostheses and the LiDAR device, although that said, in other embodiments, these axes are not stable. With respect to the concept of stable axes, these axes could be axes that would change relative to the environment shown in FIGS. 11 and 13 if the prostheses and/or the LiDAR device 2401's orientation were moved. In this regard, the mere fact that the axes 1199 and 1189 perfectly aligned with the vertical and the horizontal and the respective figures is coincidental. That said, the axes 1199 and 1189 can have orientations that are aligned 90° relative to the direction of gravity (or some other orientation providing it is stable) and/or aligned 90° relative to the long lateral axes of the recipient 99 (which is the orientation shown in the figures). The system can be configured to identify these axes and identify the angular orientation of a given component or system relative to these axes (or any other useful axes) for correlation purposes. Here, these axes 1189 and 1199 can be used from which to measure the various vectors for purposes of consistency.

In at least some exemplary implementations, the devices will have built-in coordinate systems or otherwise will have components or features that can enable a determination of the orientation of the given device. By way of example only and not by way of limitation, many smart phone devices have features that can enable a determination of the orientation of the device relative to a direction of gravity. This can be utilized to determine the orientation of the device 2401 and/or the hearing prosthesis 100, as such componentry is located therein, so as to establish a stable coordinate system. That said, in other embodiments, the arrangements can rely upon a user positioning the components in a relatively predictable manner. By way of example only and not by way of limitation, the user could place the smart phone or otherwise the LiDAR device 2401 in front of his or her face at arm's-length directly in front of him or her upon initiation of light capture, or at a 45° angle from his or her face directly in front of the user. In some embodiments, the LiDAR system can be sufficiently sophisticated to estimate a location of the LiDAR based on the captured image. By way of example, an image of a room taken at a 45° angle from the horizontal would be understood to be angled relative to its actual orientation based on common knowledge and past experience. Such machine learning or artificial intelligence or otherwise statistical analysis algorithms can be utilized to correlate the orientation of the light capture device. Indeed, the current LiDAR systems could be utilized to evaluate the orientation of the light capture device. Still, in an exemplary embodiment, as seen in FIG. 12, the LiDAR device can be placed on a cable or some fixed object at a specific orientation (e.g., standing up straight) and the orientation of the imported or otherwise preordained and utilized to obtain the data set associated with the environment in which the LiDAR system is located.

Moreover, in at least some exemplary embodiments, there is utilitarian value with respect to ascertaining the spatial orientation of the hearing prosthesis relative to the LiDAR system when the two are separate components. In some embodiments, the predetermined positioning of the LiDAR device 2401, such as at eye level in front of the recipient for example, can result in known coordinates relative to the prostheses 100 for a given recipient. In an exemplary scenario, the distance and the angle of the LiDAR device from the hearing prostheses when held and worn respectively in the normal manner that a recipient will do so, can be measured and inputted into the overall system. This stored data can then be utilized to develop as the length and angular orientation of vector 1150 relative to the hearing prosthesis 100. Thus, if the length and angular orientation of vector 1150 is known, and the length and angular orientation of vector 1199 is known, the length and angular orientation of vector 1117 can be extrapolated there from, and can be done automatically by the system.

But note that the overall LiDAR system could potentially do this in one fell swoop by obtaining an image that is 360° about the LiDAR device 2401, or otherwise sufficiently extensive to capture the objects (including humans) proximate the LiDAR device and the hearing prosthesis. In this regard, the LiDAR device can be programmed or otherwise trained to identify the hearing prosthesis and/or even the microphones of the hearing prosthesis from this obtained image and identify the orientation or otherwise extrapolate the orientation of the hearing prosthesis 100 relative to a given target object, such as the mouth of person 1110.

And note that in some embodiments, there may not be any correlation between the LiDAR device 2401 and the hearing prosthesis 100. In at least some exemplary embodiments, it is sufficient to simply position the LiDAR device 2401 in a given spatial location that has utilitarian value, and while perfect triangulation otherwise coordination between the hearing prostheses and the LiDAR system may not exist, the data set obtained based solely on the LiDAR device 2401 can be sufficient to enable utilitarian implementation of the teachings detailed herein.

Any device, system, and/or method that can enable the orientations associated with a captured image or otherwise a data set associated with objects within an environment of the system to be correlated for use with the teachings detailed herein can be utilized in at least some exemplary embodiments.

FIG. 13 presents an exemplary Cartesian coordinate system that can be utilized in some embodiments. The ramifications of FIG. 13 will not be elaborated upon except to say that instead of the spherical coordinate system of FIGS. 11 and 12, a Cartesian coordinate system can be utilized. Any coordinate system that can enable the teachings detailed herein can be utilized in at least some exemplary embodiments

Regardless of how the orientations are established, spatial relationships between the LiDAR device 2401 and/or the hearing prosthesis 100 in general, or the microphones thereof in particular, can be developed or otherwise obtained, electronically/automatically (as opposed to using a tape measure and level) in at least some exemplary embodiments, which spatial relationships can be utilized in conjunction with utilization of the hearing prosthesis 100 so as to improve the efficacy thereof relative to that which would otherwise be the case. Some embodiments can obtain spatial relationships that have an accuracy of within 30, 25, 20, 15, 10, 9, 8, 7, 6, 5, 4, 3, 2, or 1%, or any value or range of value therebetween in 0.1% increments of the actual value (the actual value being the denominator/the control), whether those values be the angular orientation and/or the length of the vector and/or the values of the X, Y and/or Z coordinates of the Cartesian coordinate system, etc. And note that values or otherwise specific unitized values need not necessarily be obtained. The coordinates and/or the data sets can simply be those of an electronic data set that has meaning only to the system. Indeed, this is concomitant with some types of artificial intelligence and/or machine learning systems, or otherwise a trained neural network, where a user thereof may not necessarily understand how exactly the device is calculating or otherwise developing the output. Accordingly, in at least some exemplary embodiments, the system is “simply” developing a data set can be utilized by the hearing prostheses for beamforming purposes for example in an exemplary embodiment, the output of the subsystem that utilizes the LiDAR is a data set that is utilized by the hearing prostheses to direct beamforming, where that data set could be incomprehensible outside of the field of use thereof. Further, the output can simply be a command to the beamformer to direct the beamformer according to the command.

Conversely, the LiDAR system can instead simply provide spatial coordinates of various objects to the prosthesis, and the microphone of the hearing prostheses can be utilized to detect sounds in the ambient environment, and based on the detection of the sound, the hearing prosthesis can determine or otherwise estimate the most likely source of the sound, and then utilize the data associated with the spatial coordinates of the various objects to determine the spatial location of the determined most likely source of the sound, and thus direct beamforming accordingly. That is, some embodiments of the teachings detailed herein utilize LiDAR coordinates otherwise spatial coordinates to better refine beamforming. In this regard, owing to echoes and reverberant sounds, or otherwise even the simple limitations of any directional determination system that relies solely upon sound to determine a directional orientation of that sound, the utilization of the spatial coordinates or otherwise the data obtained by the LiDAR system can overcome at least some of those limitations and provide for better beamforming in that a better determination of the actual origination of a given sound can be developed because the location otherwise the spatial coordinates otherwise the positioning of objects around the recipient otherwise proximate the recipient are known.

By rough analogy, a blind person often has a more highly refined sense of hearing and directionality associated with hearing to compensate for the lack of vision relative to people who have vision. This is analogous to a standard beamforming regime that utilizes sound to determine directionality of the sound. Conversely, a person having vision can often determine the source of sound simply because of the existence of the sound owing to the visual “map” that is constantly made in the person's mind based on the light captured by that person's eyes. A person knows that sound coming from the television comes from a certain direction because that person can see or have seen where the television is located. A person knows that the sound of one's wife or one's husband or one's child comes from certain direction because that person can see or otherwise has seen that person and knows where that person is located at least relatively located relative to the hearer.

Accordingly, in an exemplary embodiment, the system obtains a data set based on visual images of the environment around the recipient. This data set can include coordinates or otherwise can simply be a data set of catalog objects (object 1, object 2, object 3, object 4, etc., where objections 1 and 3 could be humans and object 2 could be a radio for example and object 4 could be a chair—the system may or may not identify the objects accordingly—it could simply enumerate them as distinct objects). Upon capturing sound by the microphones of the hearing prostheses, and initial directionality could be determined, and this initial directionality could be compared to the data set and the object closest to the determined initial direction of the sound could be declared to be the source of the sound, and then the beamforming could utilize the data of the data set to direct the beamforming towards the object declared to be the source of the sound. That is, the data set obtained from the LiDAR system would control the directionality of the beamforming.

Note further that in some embodiments, a smart system could be employed where the system extrapolates the meaning of images captured by the image sensors. By way of example only and not by way of limitation, in an exemplary embodiment, the system could determine or otherwise estimate that object 1 is a human and object 2 is a radio and object 3 is a chair. The system could immediately rule out object three is a source of sound and thus develop two directionality scenarios for the beamforming towards the two objects. And while some embodiments can utilize the directionality of the captured sound to determine which object is the likely source of the sound, other embodiments can use instead the frequency and/or the volume and/or the intonation otherwise the type of speech to determine which object is generating the sound. And this can be based on a prior catalogued data set. By way of example only and not by way of limitation, in an exemplary embodiment, the frequency of voice of people commonly encountered by the human can be considered a frequency of speech of a person, whereas frequencies of speech not common to past experience could be considered emanating from the radio for example. Volume could be utilized as a basis for distinguishing between objects. The consistency of speech or sound can be utilized to distinguish between objects. A radio or television will likely have more consistent speech than a human being that is in the same room with a recipient of a hearing prostheses. And note further, that types of people 10 be distinguished based on the characteristics of sound created thereby. For example, the LiDAR system could estimate whether or not a person is a man or a woman or a child. The frequency of the voice of that person could be utilized to determine which object is speaking.

And of course, the various concepts for determining the origin of the sound can be combined.

As will be described in greater detail below, some embodiments utilize a statistical analysis program and/or an artificial intelligence or a machine learning program or a trained neural network to identify/classify objects and/or determine the most likely object based source of sound.

Thus, as noted above, an embodiment includes having LiDAR scan objects/people in the room, the system can make a high-resolution digital 3D representations of areas in the scanning environment statically and/or dynamically. This can be that corresponding to FIGS. 9 and 10 above, for example, and from this 3D representation, the relative distance and/or spatial orientation of the recipient and/or the hearing prosthesis (or the LiDAR device) to people positioned proximate the recipient can be determined. Upon knowing the relative spatial spacing of objects/people in the environment, the system then provides guidance to control and/or steer the beamforming directionality of the hearing prosthesis to focus on the source of signals of interest, for example, the mouth of person 1110, or the center of mass of person 1110, or the head of person 1110, thus relatively better capturing the speech signals while reliably ignoring signals from other directions.

And note that many smart phones or other portable electronic devices have microphones, which microphones can be used in some embodiments to further identify sources of sound. Indeed, in an exemplary embodiment, the microphones of the smart phone or otherwise of the LiDAR system are used to capture the initial sound (in addition to the microphones of the prosthesis in some embodiments) and determine the directionality and/or the other features noted above of the sound. In this regard, the smart phone of the LiDAR system can perform all of the functions of identifying the object that is the most likely source of the sound, and can provide this determination and/or provide a control command to the prostheses for the prostheses to execute the beamforming accordingly. That is, in some embodiments, the LiDAR system/smartphone can develop the beamforming commands or coordinates autonomously and provide such to the hearing prostheses. Still, in other embodiments, it is the prostheses that ultimately develops the beamforming command requirements based on the data from the LiDAR system/smartphone.

Embodiments can include using the results from the LiDAR scan to determine room dimensions and/or geometrical shape(s) of indoor environment. This can have utilitarian value in determining room reverberation features/times. Reverberation of sound exists in many places and in different scales. Reverberation can exist in big places such as larger auditoriums and halls and smaller places like meeting rooms, living room, and kitchens. Because of the prolongation of sound reflections on the hard surfaces, audio signals including music can lose their intended clarity. Embodiments can include using the LiDAR results to mitigate such negative impact. For example, the LiDAR results can be used to develop/identify recommendations for arranging/rearranging a room with different furnishings, such as soft furnishings (e.g., the placement of curtains on the wall, coverings of the floor with carpets and/or rugs, install acoustic panels, etc.) and/or can be used to instruct the recipient to move closer to the speaker and/or even when to use assistive listening devices or what to set the devices on, etc.

With the known room size, determined via LiDAR scanning, embodiments can include algorithms that can determine/identify possible adjustments of settings of the hearing prosthesis such as the gain control, threshold of compression, compression release time, which can be executed automatically and/or dynamically, to reduce certain levels of reverberation with respect to the room size. This can be executed, in some embodiments, in combination with existing algorithms to address reverberation based on the received audio signal. More on this below.

Thus, in an exemplary embodiment, there is a system, comprising a central processor apparatus configured to receive input based on LiDAR data, wherein the central processor apparatus is configured to collectively evaluate the input to identify at least one spatial location that is more conducive to hearing with a hearing prosthesis relative to another spatial location. In some embodiments, the system is configured specifically for addressing the needs of a cochlear implant. In this regard, there are building structures/layouts that will affect the results of a cochlear implant differently than other types of hearing prostheses, and thus the system is configured to use the input based on the LiDAR data to identify at least one spatial location that is more conducive to hearing with the cochlear implant relative to another spatial location and relative to that which would be the case for another type of hearing prosthesis. Moreover, in some embodiments, the system is configured to receive input indicative of a specific recipient of the hearing prosthesis's hearing profile and the central processor apparatus is configured to collectively evaluate the input based on the LiDAR data and the input indicative of the specific recipient to identify the at least one spatial location that is more conducive to hearing with the particular hearing prosthesis relative to another spatial location.

And in some embodiments, the systems herein further include a display apparatus (such as the display 2421 or a display of a laptop or desktop computer, or a television linked to other components of the system for example) configured to provide landscape data indicative of the identified at least one spatial location that is more conducive to hearing with a hearing prosthesis relative to another spatial location.

And note that some other embodiments can be combined with sound capture devices/captured sound to evaluate/validate the identified location. Thus, the recipient could move to given locations (instructed by the system, such as by indicating on a display showing the map of the room where to stand or move), or otherwise placing the prosthesis or the smart phone or a remote microphone, etc., at the locations and capturing sound, which captured sound is used for the evaluation, and can be compared to other locations or simply to determine that the sound quality is good or acceptable.

There are thus also methods comprising obtaining the LiDAR data and evaluating the data and developing one or more acoustic landmarks based on the captured sound. And embodiments include using the developed acoustic landmarks to develop an acoustic landscape that is a two or three dimensional sound field. And in some embodiments, the acoustic landmark(s) are geographical location(s) at which a cochlear implant recipient will have a more realistic hearing percept relative to other geographical locations. In some embodiments, the data is used to develop weighted locations weighted relative to sound quality. That is, some locations can weighted more than others where such locations have superior sound quality relative to others.

Moreover, embodiments can include evaluating the data in view of data particular to a hearing related feature of a particular recipient of a hearing prosthesis and identifying a location conducive to hearing ambient sound based on the evaluation in view of the data indicative of the recipient of a hearing prosthesis.

The LiDAR data can be used and is used in some embodiments such that the action of developing one or more acoustic landmarks based on the LiDAR data includes determining a spatial location where there is minimal noise and/or reverberation interference relative to another spatial location based on the evaluation of the LiDAR data. And note further that in some embodiments, the device used to capture the image is subsequently used for purposes unrelated to hearing.

In accordance with another exemplary embodiment, there is a method, comprising capturing light or otherwise obtaining spatial data of a plurality of respectively effectively spatially separated locations of a locality, evaluating the data and developing a sound field of the locality. In some embodiments, the sound field is a three dimensional sound field, and in others, a two dimensional sound field. Methods further include scenarios where the action of developing the sound field includes evaluating the evaluated captured sound in view of data particular to a hearing related feature of a particular recipient of a hearing prosthesis and/or evaluating the evaluated captured sound in view of statistical data relating to cochlear implant recipients.

In some embodiments, the developed sound field is a first sound field of the locality and the method further includes, at a temporal location substantially different from that at which the first sound field was developed, capturing light at a plurality of respectively effectively spatially separated locations of a locality and developing LiDAR data based thereon, evaluating the second captured light (e.g., evaluating the LiDAR data) and developing a second sound field of the locality based on the action of evaluating the second captured sound. In some instances, in between the development of the first sound field and the development of the second sound field, the acoustic environment of the locality has effectively changed. Thus, embodiments can be used to account for such. And embodiments can further include identifying a recurring time period where, statistically, the sound environment is more conducive to a recipient of a hearing prosthesis relative to other time periods based on a comparison of at least the first and second sound fields.

In accordance with another exemplary embodiment, there is a method comprising receiving data indicative of LiDAR data for a plurality of spatially separated locations in a closed environment, wherein the enclosed environment has an acoustic environment such that a given sound has different properties at the different locations owing to the acoustic environment and evaluating the data to determine at least one spatially linked acoustic related data point based on one or more hearing related features of a specific hearing impaired individual. The hearing related feature of the specific individual is that the individual relies on a hearing prosthesis to hear. In some embodiments, the hearing related feature of the specific individual is that the individual has below average dynamic hearing perception at a certain sound level and/or at a particular frequency, the spatially linked acoustic related data point is a location in the enclosed environment where the effects of the below average dynamic hearing perception will be lessened relative to other locations. In some embodiments, the hearing related feature of the specific individual is that the individual has below average hearing comprehension at certain reverberation levels and the spatially linked acoustic related data point is a location in the enclosed environment where reverberation levels are lower than at other locations. Further, the hearing related feature of the specific individual can be a current profile of a variable profile of a hearing prosthesis worn by the individual.

Methods further include evaluating the data to determine a plurality of spatially linked acoustic related data points based on one or more hearing related features of a specific individual, developing a two dimensional and/or a three dimensional map of the enclosed environment presenting at least one of the acoustic related data points thereon and indicating the at least one of the acoustic related data points on the map as a recommended location for the individual to position himself or herself to improve his or her hearing in the enclosed environment. Many of the above features can be further understood in view of U.S. Patent Application Publication No. 2020/0296523 to Alexander Von Brasch and Stephen Fung, published on Sep. 17, 2020, entitled Acoustic Spot Identification.

Embodiments can include improving and/or enhancing the hearing experience for recipients in the environment/a given environment. In some embodiments, there are actions of collecting and/or understanding the information in an accurate manner, where, via the utilization of the LiDAR data, more accurate ranges, dimensions and/or shapes can be mapped out for that entire environment. These data can be fed into a modelling system to re-construct a relatively accurate 3D environment for acoustic studies customized for that individual. For instance, in real life, respective living rooms and/or bedrooms for example, of recipients is unique and different. Given such data, the modelling system is used make use of the real data to construct an augmented reality which is unique and familiar to that individual for training. Overall, being trained in a comfortable and familiar environment could relax and make the recipients less anxious. In essence, the system can learn how to accurately correlate the spatial objects/environment with its acoustic properties.

Indeed, in an embodiment, there exists the creation of a virtual reality experience and/or an augmented reality experience using the LiDAR data. In an exemplary embodiment, there is a method, comprising obtaining access to a virtual reality system, activating a cochlear implant (or any other implant detailed herein) such that the cochlear implant evokes a hearing percept based on first input and receiving second input from the virtual reality system in temporal proximity with the evocation of the hearing percept based on the first input, wherein the cochlear implant (or whatever implant) evokes a hearing percept based on the first input, and the second input is correlated with the first input, wherein the virtual reality system provides output to the recipient based at least in part on data from the LiDAR data. The first input is generated by the virtual reality system and/or the second input is visual input. Here, the virtual reality system can present images with which the recipient is familiar where the images are created from the LiDAR data were otherwise based on the LiDAR data. Thus, embodiments include utilizing the LiDAR systems disclosed herein to create the visual portion of the virtual reality experience.

Embodiments include a system, comprising a virtual reality sub-system and a hearing prosthesis, wherein the virtual reality sub-system is configured to provide first output to the hearing prosthesis and/or work in conjunction with the hearing prosthesis, the hearing prosthesis is configured to evoke a hearing percept in a recipient thereof based on the first output, the virtual reality sub-system is configured to simultaneously provide second output to the recipient of the hearing prosthesis to evoke a second sensation different from hearing (e.g., a visual sensation developed based on the LiDAR data). In some embodiments, the second sensation is a sensation that results from the real-life physical phenomenon that results in the hearing percept. In some embodiments, the system is configured to train the recipient in sound-object association and/or sound direction association by evoking a hearing percept of a sound produced by an object or from an object's direction and presenting an image of the object in is environment (e.g., an environment presented as a visual image based on the LiDAR data) using the virtual reality sub-system.

In some embodiments, the system is configured to train the recipient in sound-location association and/or sound-direction association by evoking a hearing percept of a sound and providing a virtual-reality stimulus to the recipient indicative of a directional origin of the sound. In some embodiments, the system can train the recipient in the voice-recognition and/or object recognition. There can be utilitarian value with respect to presenting these objects in an environment with which the recipient is familiar, which environment can be developed utilizing the LiDAR data.

Moreover, in some embodiments, the system is configured to train the recipient in reverberant sound recognition by evoking a hearing percept of a sound and providing an image of an environment in which the sound having a given reverberation is heard, and this image can be based on the LiDAR data. Moreover, the system can be configured to train the recipient to recognize and/or differentiate between sound types by evoking a hearing percept of sound and providing an image of the sound making device as located in a given environment in real life. Again, the environment being extrapolated from the LiDAR data.

Moreover, there is a method, comprising a first action of evoking an artificial hearing percept in a recipient of a hearing prosthesis based on input indicative of a first sound and a second action of receiving first visual input, which first visual input is correlated with the first sound, wherein the second action is executed in effective temporal correlation with the first action and repeating the first and second actions, thereby improving the recipient's ability to recognize the first sound, wherein the visual input is based on the LiDAR data. The first visual input originates from an artificial source and/or the first visual input is generated by a virtual reality system.

Embodiments can include a method, comprising providing a recipient of a cochlear implant hearing prosthesis (or another type of prosthesis detailed herein) a first sound and non-sound stimulation scenario, wherein an artificial hearing percept of the first sound is evoked with the hearing prosthesis and providing the recipient of the hearing prosthesis a second sound and non-sound stimulation scenario wherein an artificial hearing percept of the second sound is evoked with the hearing prosthesis, wherein the non-sound stimulation is a virtual reality initiated stimulation, and the provided scenarios train or retrain the recipient to associate the respective sounds with the respective non-sound stimulation, the non-sound stimulation being developed from the LiDAR data. And in view of the above, it can be seen that in at least some embodiments, there is a method of training or retraining a recipient of a cochlear implant and/or another type of hearing prosthesis detailed herein to recognize and/or distinguish sounds using virtual reality, wherein the virtual reality is implemented based at least in part on the LiDAR data. The action of training or retraining includes providing audio-visual scenarios using virtual reality of increasing complexity as the recipient learns to recognize and/or distinguish sounds of the less complex scenarios. The action of training or retraining results in the recipient distinguishing between different species of sounds of a genus of sounds, wherein the recipient could not distinguish between those different species of sounds prior thereto.

In some embodiments, the action of training or retraining results in the recipient being able to at least one of recognize a given sound in different reverberant environments or distinguish between a sound in different reverberant environments, wherein the recipient could not at least one of respectively recognize the sounds in the different reverberant environments prior thereto or distinguish between the sound in different reverberant environments prior thereto.

Thus, embodiments can use the LiDAR data to develop a personalized rehabilitation and/or habilitation environment model, and using that model for habilitation and/or rehabilitation of hearing using the hearing prosthesis and the model.

Note that the LiDAR systems herein can be combined with other functionalities that also can be based on LiDAR. For example, the LiDAR system can be extended to perform an alarm function so as to warn the user of potential dangers. This can be implemented by having the system detecting specific objects or environments that are hazardous, based on the LiDAR data, and providing an audible warning to the user, which can be implemented through the hearing prosthesis (or another type of prosthesis) for example. Indeed, the warning can be solely generated by the prosthesis, such that it cannot be heard or at least is not head by others, thus providing a level of discretion.

Embodiments thus include a system, comprising a light sensor and/or a sonic sensor and/or a radio wave sensor. Again, while embodiments described above have typically been directed towards the utilization of light capture devices of smart phones that can be utilized to implement the LiDAR features, embodiments are not so limited. As noted above, sonar type devices or radar type devices can be utilized. Thus, some embodiments include systems that have different types of sensors as just noted. At least many embodiments will utilize sensor(s) that capture images used for a LiDAR device. The system further includes a processor configured to receive input based on light and/or sonic frequency reflection (in some embodiments, ultrasonic frequencies are utilized) and/or radio wave reflection captured by the respective sensor. Note that in some embodiments, there will also be an emitter of light, sonic energy and/or radio waves in the system, and thus some embodiments are active sensors instead of passive sensors (LiDAR can be considered passive). And note that the infrared spectrum can be utilized as well. The system is further configured to analyze the received input and develop data, which can be a dataset or can be a simple signal, usable to control a sensory prosthesis based on the received input. This data can be a prosthesis control dataset, or can be data useable by a prosthesis. The data can be developed by the system that includes the LiDAR system, by the prostheses, or by some other component, such as, for example, a component completely remote from the sensors and/or the hearing prostheses, such as a remote data center or a remote server that is accessible via the Internet or some other means of electronic communication.

The data developed by the system can include instructions for beamforming (thus prosthesis control data). In this regard, the data can be a dataset that can include weighting values for weighting the microphones of the hearing prostheses or other sound capture device that utilized with the system to steer the microphones in the desired beamforming direction. This is described above with respect to utilizing the LiDAR data to develop a two or three dimensional model of an environment and to identify objects therein or otherwise identify the position of objects therein to better steer the beamforming. The data need not necessarily include the weighting values, but instead can simply include instructions to control the prosthesis to achieve the ultimate weighting. Of course, other types of control features can be included in the data. Anything that can be usable for beamforming can be included in the data.

But note that in other embodiments, the data developed by the system can be control settings for settings of the prosthesis, such as user control settings (although others may not be user controlled, at least not per se—for example, the stimulation times of the current from the electrodes of the cochlear implant or the frequency of the alternating current from the electrode array are not typically user controlled settings, but can be adjusted by an audiologist for example). By way of example only and not by way of limitation, the data can be gain control data, threshold of compression data, compression release time, etc. The data can be anything that can be used to adjust the hearing prosthesis to improve the efficacy thereof. As noted above, some embodiments include the adjustment of the prostheses based on the LiDAR scanning to automatically and/or dynamically reduce the results of certain levels of reverberation in an effective manner.

Some embodiments can utilize the LiDAR data as a basis for masking and/or noise cancellation regimes implemented by a hearing prosthesis. The obtained LiDAR data can be utilized to identify types of objects in an environment, and mask or cancel or at least partially cancel sound emanating from some objects in the environment that are identified. This could entail advanced image recognition features that can be executed automatically. In some embodiments, the masking or canceling could be part of a method where the system identifies an object in an environment and asks the recipient to confirm that that object should be masked or canceled. The system can remember past examples of such and can automatically implement such in the future, at least after providing an indication of the recipient that such will happen or has happened.

Moreover, by having a model including vectors and/or distance values to various surfaces in the environment, the system can be utilized to determine times of arrivals of sound based on the known value for the speed of sound, and otherwise can utilize the LiDAR data for reverberant processing purposes to at least partially mitigate the effects of reverberant sound. Without at least approximate distances to various surfaces within the environment and/or sound sources within the environment, reverberant processing would be less effective.

And note that the developed data can be a control signal for example. The system can develop a control signal that is sent to the device that controls the microphones, or the gain setting, or the attack times for feedback control algorithms, or the compression thresholds, etc. With respect to beamforming, the control signal can be used to control the microphones to, for example, weight the microphones relative to one another to steer the directionality of the sound capture apparatus in accordance with the control signal.

In some embodiments, the system includes the prosthesis, which can be a hearing prosthesis or a light prosthesis, such as a retinal implant with respect to the latter (here, the system can correspond to system 210 above, for example). It is noted that in some embodiments, the system does not necessarily include the prosthesis. In this regard, the system can be utilized to simply develop the data. Again, in some embodiments, the developed data is a control signal, and, where the system includes the prosthesis, circuitry of the prosthesis is in signal communication with a component of the system that generates the control signal. This can be by a wireless link for example between the smartphone 2401 and the hearing prosthesis, where the control signal can be communicated in real time.

Again, in some embodiments, the prosthesis is a hearing prosthesis, such as a cochlear implant, a middle ear implant, a bone conduction device and/or a conventional hearing aid (the prosthesis can be a hybrid device, which includes any two or more of those prostheses).

Consistent with the teachings above, the system includes the light sensor and the input is based on light captured by the light sensor, and the light sensor is part of a LiDAR sub-system that is part of the system. In some embodiments, the processor is configured to develop second data indicative of a two-dimensional and/or a three-dimensional digital model of the area and/or volume, respectively, proximate the respective sensor. This can be the model itself, or can be a dataset for example based on the model (e.g., it include vectors and/or distances, and/or angles, etc., to identified points in space—thus, it is not a model of the area, but a model based on the model of the area). In this embodiment, the processor is configured to develop the data usable to control the prosthesis based on the second data. For example, it can use the model data to identify a beamforming control signal or dataset, or can use the aforementioned vectors and/or distances to control the beamforming. The prosthesis or another part of the system can have a program that can analyze the model and/or the vectors, etc., and develop the beamforming control based thereon.

Corollary to the above is that the processor can be configured to develop second data indicative of distance of objects proximate the respective sensor and the processor is configured to develop the data to control the prosthesis based on the second data. Again, in some embodiments, the developed data is a dataset, while in other embodiments, it is a control signal.

Embodiments can include a system and/or simply an embodiment that includes a non-transitory computer readable medium having recorded thereon, a computer program for executing at least a portion of a method, the computer program including code for analyzing first data based on data captured by a light sensor and/or sonic sensor and/or radio wave sensor (this can be the LiDAR software, for example, or any comparable software that can enable the teachings herein). The medium further includes for example, code for quantifying and/or qualitatively identifying a sensory prosthesis performance impacting feature of an environment in which the light sensor and/or sonic sensor is located. This can be a distance to a person or sound source, an angle/orientation of such to a microphone of the system or the sensor, features of a room, such as hard surfaces, angles of surfaces, soft surfaces, spatial positioning of surfaces, etc.

Again, in some embodiments, the feature is a distance to an object in the environment from the light sensor and/or sonic sensor and/or a radio wave sensor and/or a sensor of the sensory prosthesis (e.g., a microphone of the hearing prostheses or a microphone of the smart phone for example). Concomitant with the above, in an exemplary embodiment, the feature is an angle to an object in the environment from the light sensor and/or the sonic sensor and/or the radio wave sensor and/or a sensor of the sensory prosthesis or another sensor of a system of which the sensory prosthesis is a part. There can be two angles in two planes for example (one for the horizontal plane and one for the vertical plane), or in some embodiments, there can be simply a horizontal plane angle or a vertical plane angle. Moreover, it is noted that that the concept of angle is not limited to spherical coordinates. Angles can be implied in cartesian coordinates. And note further that polar coordinates can be used in some embodiments.

In some embodiments, the sensory prosthesis performance impacting feature is a feature that is accommodated by a real-time adjustment to the sensory prosthesis during use of the sensory prosthesis. This can be beamforming, gain adjustment, etc.

And note that by real-time, it is meant that the adjustment can be executed in relatively close temporal proximity to the action of analyzing the first data based on data captured by the given sensors. This as compared to other embodiments where, for example, a LiDAR system is utilized to obtain a model of the room, and then a habilitation and/or rehabilitation regime is developed based on that model. The model could be used hours or days or weeks after the model is developed and/or after the images are captured to develop the LiDAR data.

In some embodiments, the medium includes code for automatically controlling the sensory prosthesis based on the identified feature.

In an exemplary embodiment, a given time period that can encompass two or more of the actions of obtaining data with a light and/or sonic and/or radio wave sensor, analyzing data obtained data and quantitatively and/or qualitatively identifying the performance impacting feature or adjusting the prostheses and/or automatically controlling the prostheses based on the identified feature can extend is less than 5, 4, 3, 2, 1, 0.75, 0.5, 0.25, 0.1, 0.05 seconds, or any value or range of values therebetween in 0.01 second increments.

In some embodiments, the computer program is a dual purpose hearing habilitation and/or rehabilitation program and a real time hearing perception improvement program. That said, in some embodiments, the various parts of the computer program lie on the same computer.

While the embodiments above have typically focused on automated systems that in automatically adjust the given component of the system in a manner that is generally autonomous relative to a user recipient, albeit in some embodiments, the user or recipient can enable the automatic functionality and/or disable the automatic functionality of such, and in some embodiments, there is also code for providing data to a human pertaining to the identified feature. This can enable the recipient to at least understand what is going on with the environment in which the recipient finds himself or herself, or, in the oven automated adjustment to the prosthesis, inform the recipient of what the prostheses is doing or at least why the prostheses is doing something and/or provide the recipient or user with information so that the recipient or user can take affirmative action with respect to making an adjustment of the prostheses to account for the identified feature.

And note that some embodiments include code that provides data to a human based on the identified feature. In this regard, the information need not necessarily be the identified feature, but could be information such as a recommended change to a setting or that the recipient take a certain action, such as, for example, look in one direction or another direction or tilt his or her head in a given manner, or increase or decrease in volume for example were adjust a gain setting etc. This information can be conveyed via the smart phone or the portable handheld device, or could be conveyed via a message provided by the prosthesis, whether that be via a hearing percept that is evoked by the prostheses or a like precept, such as a text message, evoked by the light prosthesis.

In some embodiments, the data provided to the human could be the two-dimensional or three-dimensional model created by the LiDAR system, where the model is annotated or otherwise includes enumeration that can be useful to the recipient to improve his or her experience with the prostheses. By way of example only and not by way of limitation, the three-dimensional image could include an annotation such as, “look here” or “point head here” where the location could be the mouth of a speaker or could be a location potentially on top of the head of a speaker, or somewhere else, where, for example, owing to the reverberations in a given environment, tilting one's head ever so slightly away from the direction that the recipient might otherwise expect to lock or point the prosthesis towards can have utilitarian value. In this regard, the information provided to the recipient could be counterintuitive information.

But going back to the concept of utilizing the LiDAR data, in an exemplary embodiment, the computer program can include code for determining a 2D and/or 3D field in which the respective sensor is located, such as from light captured from cameras of a LiDAR system, or from a radar return captured by a radar transducer, or sonar return captured by a sonar transducer, etc., wherein the code for identifying the feature uses the determined 2D and/or 3D field to identify the feature. The code can be the code used in a LiDAR system.

Embodiments can utilize the models developed from the LiDAR data or the other data to evaluate the acoustics of a given room or space or area and otherwise estimate the soundfield of the actual area that the model represents. In an exemplary embodiment, the systems herein include image recognition and/or structure recognition capabilities that can identify the types of structure and/or the types of objects (including people) in the model. For example, the systems herein can identify a table or a floor that is a hard floor versus a carpeted floor or a bed or a sofa or a wooden chair versus a cushioned chair, etc. The identified objects can be compared to a database to obtain acoustic data associated there with, including statistical data relating to how a given object is positioned in a similarly situated room. Additional details of this will be provided below. But briefly, in at least some embodiments, the system can identify a table and identify for example a relevant dimension or two of the table, and then access a database having acoustical features associated with, for example, a table of a given size in a given room of a given size, and then utilize those acoustical features to develop acoustical data for the given area.

Embodiments thus include code for determining an estimated sound field in which the respective sensor is located, wherein the code for identifying the feature uses the determined estimated sound field to identify the feature. That is, the teachings detailed herein can be utilized to establish a sound field in real-time or close thereto by harnessing the data captured by the sensor(s) in a given sound environment. The embodiments herein can provide the ability to establish a true sound field, as opposed to merely identifying the audio state at a single point at a given instant. In this regard, the teachings detailed herein can be utilized to provide advice to a given recipient as to where he or she should go in the enclosed volume, as opposed to whether or not a given location is simply good or bad.

Note further that in some embodiments, sound capture devices of the prosthesis or of the smart phone or other device for example can be utilized to capture sound within a room rather area that forms the basis for the models detailed herein, and this captured sound can be digitized in a statistically meaningful manner, and then used with the given model for the given area to develop a sound field related to a given area. This can be used to later develop sound fields for other modeled areas without obtaining sound.

In an exemplary embodiment, there is a system that is configured to locate an optimal hearing spot/point/location/area for the recipient. In an exemplary embodiment, this is the optimal hearing spot/point/location/area, and in other embodiments, is one of a plurality of such. The system is configured to analyze output from the sensor(s) (e.g., the LiDAR cameras) that are received or otherwise divided from the various devices, and use this information to form one-dimensional, two-dimensional and/or three-dimensional sound field of the environment in which the sensors are located. This is done, in an exemplary embodiment, in real-time, while in other embodiments, it is not done in real time. In an exemplary embodiment, the system is configured to receive a recipient's hearing profile as part of the criteria for locating and deciding whether the selected acoustic spot/zone would be utilitarian (e.g., ideal) for a given particular individual.

Alternatively, and/or in addition to this, consistent with the teachings detailed above, in an exemplary embodiment, the action of developing the sound field of the locality can include the action of evaluating the evaluated modeling data in view of statistical data relating to cochlear implant users and/or other types of hearing prosthesis users. In this regard, there is data available and/or there is data that can be developed over a statistically significant group of cochlear implant users that can enable statistically significant factors to be deduced based there from. In this regard, the sound field of the locality can be developed so as to identify locations that are conducive or otherwise favorable to improving the hearing experience of a statistically normal cochlear implant user. By way of example only and not by way of limitation, it is known that cochlear implants have an electrical sound/synthesized sound. Some may consider the sound to be analogous to a breathless person speaking in a hushed manner. A location in the locality or a plurality of locations in the locality can be identified where the captured sound will be more compatible with the hearing percept evoked by a cochlear implant relative to other locations. By way of example only and not by way of limitation, a location where sounds are more pronounced and otherwise have little reverberant sound therein or otherwise minimize reverberant sound relative to other locations can be identified when developing the sound field of the locality. Of course, in some embodiments, the sound field of the locality can simply correspond to indicators that indicate that such a location is useful for cochlear implant users. Of course, in some embodiments, the action of evaluating the modeling data can be executed in view of statistical data relating to other types of hearing implant recipients, such as, for example, middle ear implant recipients and/or bone conduction recipients and/or normal conventional hearing aid recipients, etc. Moreover, in some embodiments, the action of evaluating the modeling data can be executed in view of statistical data related to a specific model or design of a given implant. By way of example only and not by way of limitation, in an exemplary embodiment, if the cochlear implant is a so-called small or short cochlear implant electrode array design configured to preserve residual hearing, the action of developing a sound field of the locality correspond to providing indicators of locations where a recipient utilizing such design and/or model will have a better hearing experience relative to other locations. Indeed, in an exemplary embodiment, the sound field can indicate locations for total electric hearing persons as well as for persons that have partial electric hearing in a given ear.

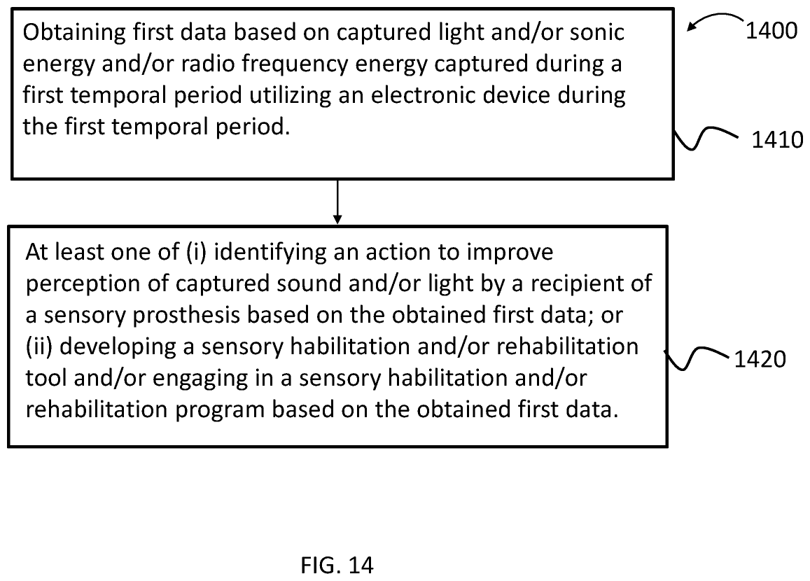

FIG. 14 presents an exemplary flowchart for an exemplary method, method 1400, according to an exemplary embodiment. Method 1400 includes method action 1410, which includes the action of obtaining first data based on captured light and/or sonic energy and/or radio frequency energy captured during a first temporal period utilizing an electronic device during the first temporal period. This could be executed by utilizing a LiDAR capable smart phone for example or scanning an area with a camera capable of obtaining images that can be utilized to develop a LiDAR model, and transferring those images or data based on those images to a computing device that can develop a model based on those images. This could also be executed by simply obtaining the images or data based on the images or otherwise even obtaining the results of the LiDAR system, which can be the LiDAR model of a given area. That is, method action 1410 need not necessarily require the actual capturing of the images of a room.

And note that method action 1410 can include accessing a data set or the model or accessing a data set based on the model that can enable the teachings detailed herein. For example, method action 1410 can be executed by engaging the beamforming algorithm where the beamforming algorithm accesses first data that is based on the captured light or energy to implement the beamforming, where the action of obtaining the hearing prosthesis or the smart phone or other handheld device or otherwise accessing the database corresponds to method action 1410.

Method 1400 also includes method action 1420, which includes at least one of (i) identifying an action to improve perception of captured sound and/or light by a recipient of a sensory prosthesis (e.g., a hearing prosthesis or a retinal prosthesis, respectively) based on the obtained first data or (ii) developing a sensory habilitation and/or rehabilitation tool and/or engaging in a sensory habilitation and/or rehabilitation program based on the obtained first data.

With respect to “i,” this can include the prosthesis or the smart device or a remote device remote from the prostheses but in communication with the recipient via the Internet or via cell phone for example identifying an adjustment to the hearing prostheses settings, such as a gain setting or an attack time for noise cancellation, etc., and recommending that change in setting to the recipient. This can also include the prostheses or other pertinent device automatically adjusting the prostheses accordingly. Further, consistent with the above-noted teachings relating to beamforming, this can include changing the beamforming or otherwise directing the microphones to capture sound towards one area over another area. In this can all be done in real-time with respect to the action of capturing the light or sonic energy or radiofrequency energy, etc. that was used to develop the model for example. Thus, the action identifying an action to improve perception of captured sound and/or late can be executed during the first temporal period or during a second temporal period after the first temporal period, where the second temporal period could be more than 1, 2, 3, 4, 5, 6, 7, 8, 9, 10, 11, 12, 13, 14, 15 minutes or hours or days after method action 1410 and/or after the action of obtaining the data from the sensors/obtaining the actually capturing the light and/or sonic energy and/or radiofrequency energy.

Section “i” of method action 1420 can correspond to any of the adjustments and/or controls etc., applied to the prosthesis detailed above or others.

It is noted that the teachings detailed herein are not limited to fixed or static objects—indeed, owing to the abilities in some embodiments to capture images of an area and develop a model and then implement the teachings detailed herein based on that model in short order, embodiments include utilizing the teachings detailed herein in environments that are not statically stable otherwise can change in short order, such as, for example, a bus or train where people will come in and out of an area, or an office where a meeting is held, or a classroom, etc. Embodiments thus can include executing one or more of the actions detailed herein, such as the action of capturing the light, the action of developing the model (e.g., the LiDAR model) based on the light, and then identifying an action to improve perception of captured sound and/or light by the recipient and/or implementing that action (including automatically) all within time frames less than and/or equal to 15, 14, 13, 12, 11, 10, 9, 8, 7, 6, 5, 4, 3, 2.5, 2, 1.5, 1, 0.75, 0.5, 0.25, 0.1 minutes or any value or range of values therebetween in 0.01 minute increments.

Method action 1420 also includes a second subsection “ii” that includes the action of developing a sensory habilitation and/or rehabilitation tool and/or engaging in a sensory habilitation and/or rehabilitation program based on the obtained first data. In this regard, as noted above, in an exemplary embodiment, a virtual reality experience can be implemented where at least the visual images can be based on the LiDAR data or the model that is developed by the data captured by the sensors, etc. This virtual reality experience could be part of a habilitation and/or rehabilitation tool developed explicitly for a given recipient, which would be different than that for other recipients, at least because, for example, other recipients may have other living accommodations and/or work accommodations and/or a different type of car or ride in a different type of train or a different type of bus, etc. of course, other types of habilitation and/or rehabilitation tools can be developed utilizing the first data based on captured light or the other types of energy. Again, in an exemplary embodiment, it could be that the data is utilized to instruct the recipient where to sit in a room relative to other locations during training of sound and/or word recognition in a given room. By analogy, hearing training can be executed in a controlled environment, and thus the identification of a superior location relative to others is simply one aspect of that controlled environment, this is differentiated from, for example, hearing during normal life where the position of the recipient is less controllable.

And this is where, for example, in some embodiments, the action of developing the habilitation or rehabilitation tool and/or engaging in the sensory habilitation and/or rehabilitation program can be executed at a greater temporal distance from the first temporal distance then some embodiments where the teachings herein are used in real-time or near real time. Many days or weeks could elapse from the time of obtaining the data from the energy sensors to the time of developing the tool or the engagement of the rehabilitation and/or habilitation program. But note also that the greater temporal time periods are not mutually exclusive to training or tools. In an exemplary embodiment, the sensor data can be utilized to develop a model, and then that model can be utilized in real-time many days or weeks or even months after that model is developed to implement the identifying action and/or the adjustment action.

Returning back to method 1410, where the method 1400 includes the action of identifying the action to improve perception of captured sound and/or light by a recipient of a sensory prosthesis based on the obtained first data, the method further includes capturing sound and/or light during a second temporal period subsequent the first temporal period but spaced from the first temporal period by a third temporal period so that the first and second temporal periods are not real time or near real time, utilizing at least one hearing prosthesis, wherein the captured sound and/or light is captured in a manner at least partially based on the identified action. Here, that can correspond to implementing beamforming based on the identified action. Conversely, again where method 1400 includes identifying the action to improve perception of captured sound and/or light by a recipient of a sensory prosthesis based on the obtained first data, there is the action of capturing sound and/or light during the first temporal period and/or during a second temporal period subsequent the first temporal period but where the first temporal period and the second temporal period are sufficiently close that the first and second temporal periods can be considered real time or near real time, utilizing at least one hearing prosthesis, wherein the captured sound and/or light is captured in a manner at least partially based on the identified action.

Again, where method 1400 includes the action of identifying the action to improve perception of captured sound and/or light by a recipient of a sensory prosthesis based on the obtained first data, there can be the further action of capturing sound and/or light during the first temporal period and/or during a second temporal period subsequent the first temporal period utilizing at least one hearing prosthesis, and the method can further include evoking a sensory percept based on the captured sound and/or light at least partially based on the identified action. This can entail, for example, adjusting a gain, adjusting a threshold level, adjusting a noise cancellation attack time, or any of the adjustments detailed herein. And again, the first and second temporal periods can be separated so that these are done in real time and/or near real time or not in either.

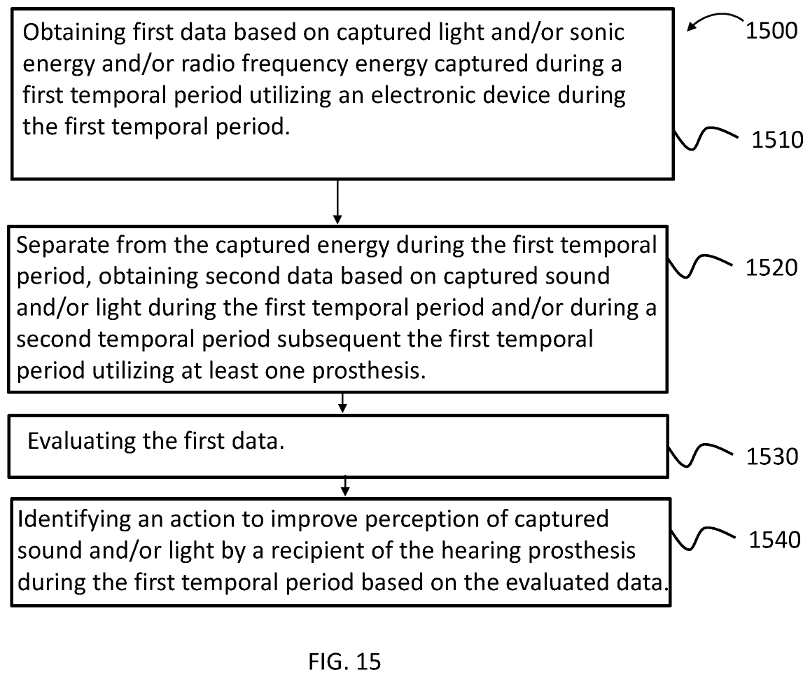

FIG. 15 presents an exemplary flowchart for an exemplary method, method 1500, according to an exemplary embodiment. Method 1500 includes method action 1510, which includes obtaining first data based on captured light energy and/or sonic energy and/or radio frequency energy captured during a first temporal period utilizing an electronic device during the first temporal period. This could be executed by utilizing a LiDAR capable smart phone for example or scanning an area with a camera capable of obtaining images that can be utilized to develop a LiDAR model, and transferring those images or data based on those images to a computing device that can develop a model based on those images. This could also be executed by simply obtaining the images or data based on the images or otherwise even obtaining the results of the LiDAR system, which can be the LiDAR model of a given area. That is, method action 1510 need not necessarily require the actual capturing of the images of a room.

Method 1500 further includes method action 1520, which includes, separate from the captured energy during the first temporal period, obtaining second data based on captured sound and/or light during the first temporal period and/or during a second temporal period subsequent the first temporal period utilizing at least one prosthesis. Method 1500 also includes method action 1530, which includes evaluating the first data, and this can be done automatically by the hearing prosthesis and/or the smart phone or a remote device miles away from the recipient.

Method 1500 also includes method action 1540, which includes identifying an action to improve perception of captured sound and/or light by a recipient of the hearing prosthesis during the first temporal period based on the evaluated data.

At least some exemplary embodiments according to the teachings detailed herein utilize advanced learning signal processing techniques, which are able to be trained or otherwise are trained to detect higher order, and/or non-linear statistical properties of signals. An exemplary signal processing technique is the so called deep neural network (DNN). At least some exemplary embodiments utilize a DNN (or any other advanced learning signal processing technique) to process a signal representative of captured sound, which processed signal is utilized to evoke a hearing percept. At least some exemplary embodiments entail training signal processing algorithms to process signals indicative of captured light or the reflected sonic energy and/or radio frequency energy. That is, some exemplary methods utilize learning algorithms or regimes or systems such as DNNs or any other system that can have utilitarian value where that would otherwise enable the teachings detailed herein to analyze the data captured by the sensors (e.g., the LiDAR sensors). In embodiments, the teachings detailed herein can be utilized to analyze or otherwise process a signal that is based on captured light and, based on the analysis, execute one or more of the teachings herein.

A “neural network” is a specific type of machine learning system, and embodiments include using a neural network to analyze the data captured by the sensor. Any disclosure herein of the species “neural network” constitutes a disclosure of the genus of a “machine learning system.” While embodiments herein focus on the species of a neural network, it is noted that other embodiments can utilize other species of machine learning systems accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method, at least one that constitutes analysis, constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system that can have utilitarian value) will be described.

It is noted that in at least some exemplary embodiments, the DNN or the product from machine learning, etc., is utilized to achieve a given functionality and/or method action as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

Accordingly, embodiments can use a DNN or a product from machine learning or other types of artificial intelligence systems to analyze the data based on data from the sensors (which could be the data directly from the sensors, or data compiled based on the data from the sensors, which can be a spatial model for example) and/or the identifying of the action to improve perception of captured sound and/or light (whether that be the beamforming “command” or where the beamforming should be pointed, or a setting of the prosthesis that is adjustable).

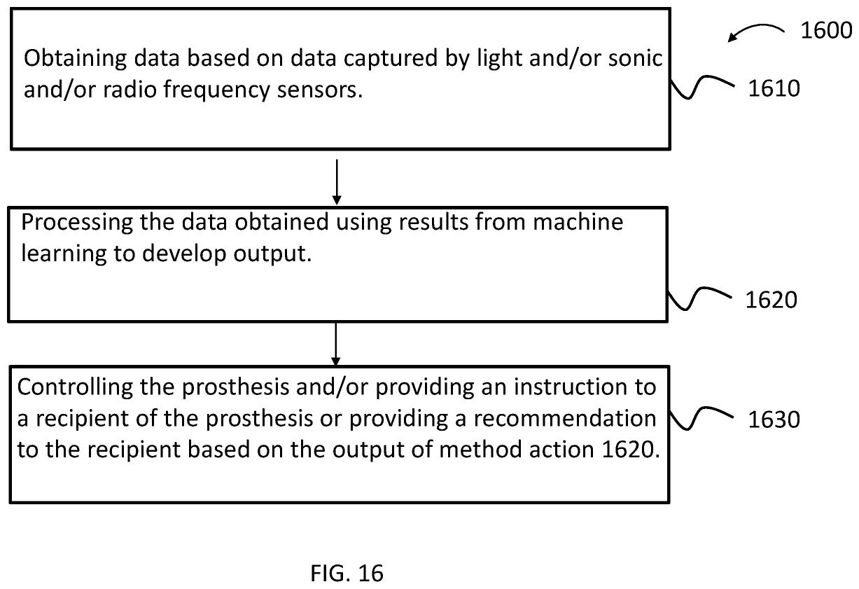

FIG. 16 depicts an exemplary flowchart for an exemplary method, method 1600, of utilizing a product of and/or from machine learning, such as a DNN, according to an exemplary embodiment. Method 1600 includes method action 1610, which includes obtaining data based on data captured by light and/or sonic and/or radio frequency sensors. In an exemplary embodiment, the obtained data is a signal containing video content. In an exemplary embodiment, this is the signal that comes from the light capture device of the prosthesis or of the smart phone, etc. In an exemplary embodiment, this is the signal that comes from a digital and/or analog device. In an embodiment, this is a dataset based on the sensor captured data. In an exemplary embodiment, the data of method action 1610 can be a data set based on the previously captured light. For example, this could be the results of the LiDAR system and otherwise could be the model of the area of interest.

Method 1600 further includes method action 1620, which includes processing the data obtained in method action 1610 to develop output. In an exemplary embodiment, this can correspond to processing the raw signals from the sensors or data stored in memory devices based on the raw signals, and thus the data is the data obtained in method action 1610. Further, this action of method action 1620 can correspond to processing a modified signal or even a new signal that is ultimately based on the signals from the light sensors for example. In an exemplary embodiment, the action of processing can occur after the filtering. In an exemplary embodiment, the product is a chip that is fabricated based on the results of machine learning. In an exemplary embodiment, the product is a neural network, such as a deep neural network (DNN). The product can be based on or be from a neural network. In an exemplary embodiment, the product is code. In an exemplary embodiment, the product is a logic circuit that is fabricated based on the results of machine learning. The product can be an ASIC (e.g., an artificial intelligence ASIC). The product can be implemented directly on a silicon structure or the like. Any device, system and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

The output could be the identified action to improve the perception of the captured sound and/or light.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish sensory prosthetic devices or supplemental components utilized with sensory prostatic devices (e.g., a smart phone), implement at least some of the teachings herein.

As noted above, method action 1620 entails processing the data utilizing a product of machine learning, such as the results of the utilization of a DNN, a machine learning algorithm or system, or any artificial intelligence system that can be utilized to enable the teachings detailed herein. This as contrasted from, for example, processing the data utilizing general code or utilizing code that not from a machine learning algorithm or utilizing a non AI based/resulting chip, etc. In an exemplary embodiment, a typical cochlear implant processes a signal from a microphone and subsequently provides the results of that processing to a stimulation device that stimulates various electrodes in a weighed manner. This processing is typically done by a sound processor which includes filter banks that simply divides up an input signal into separate filter groups or filter bins. This is not the utilization of a machine learning algorithm. That said, it is noted that in some embodiments, this division can be executed utilizing results from machine learning (e.g., a trained DNN, on whatever medium that can enable such, such as a chip).

Again, in an exemplary embodiment, the machine learning can be a DNN, and the product can correspond to a trained DNN and/or can be a product based on or from the DNN (more on this below).

FIG. 1600 further includes method action 1630, which includes controlling the prosthesis and/or providing an instruction to a recipient of the prosthesis or providing a recommendation to the recipient based on the output of method action 1620. In an exemplary embodiment where method 1600 is executed utilizing a cochlear implant, this can be to control the frequency and/or the length of stimulation, or adjust the gain, etc.

In an exemplary embodiment, method action 1610 and/or method action 1620 can be executed by a separate device, such as by way of example only and not by way of limitation, device 2401, and/or a remote device or the like, such as a device that is in communication with the tele-coil).

Consistent with the teachings detailed above, in an exemplary embodiment, all of the actions associated with method 1600 are executed by a self-contained body worn sensory prosthesis. Still, in other embodiments, such as where processing power is constrained, some of the actions are executed by device that is separate from the self-contained body worn sensory prosthesis, and the results of those actions are communicated to the sensory prosthesis so that the sensory prosthesis can evoke a sensory percept based on the results of that action.

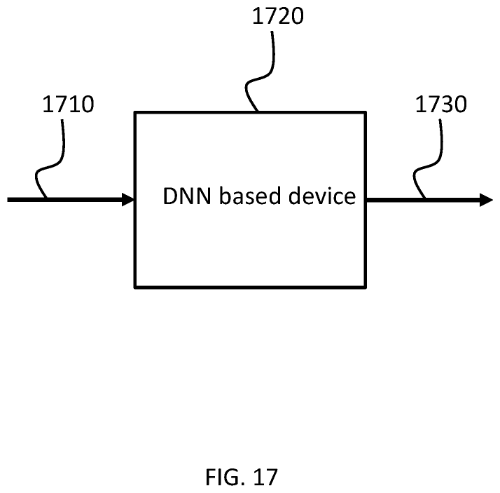

FIG. 17 depicts an exemplary conceptual functional black box schematic associated with method action 1720, where the signals from the energy sensors or data based on energy captured by the energy sensors 1710 is the input into a DNN based device 1720 that utilizes a trained DNN or some other trained learning algorithm or trained learning system (or the results thereof-in an exemplary embodiment, the product of-machine learning-as used herein can correspond to a trained learning algorithm or trained learning system as used in operational mode after training has ceased and product of machine learning can correspond to a product that is developed as a result of training-again, this will be described in greater detail below), and the output is a signal 1730 that is provided to a prosthesis or to a recipient. In this exemplary embodiment, device 1720 can be part of a hearing prosthesis or a light prosthesis, and in other embodiments, it can be a separate device (e.g., embodied in a smart phone or another device).

It is noted that in at least some exemplary embodiments, the input 1710 comes directly from the sensors, while in other embodiments, this is not the case. Input 1710 can correspond to any input that can enable the teachings detailed herein to be practiced providing that the art enables such. Thus, in some embodiments, there is no “raw light” input into the DNN. Instead, it is all pre-processed data. Any data that can enable the DNN or other machine learning algorithm or system to operate can be utilized in at least some exemplary embodiments.

It is noted that at least some embodiments can include methods, devices, and/or systems that utilize a DNN inside a cochlear implant system or another type of hearing prosthesis as detailed above or another type of prosthesis and/or along with such a system for the purposes of generating the above noted output. In some embodiments, a neural network, such as a DNN, is used to directly interface the input data, process the data via its neural net, and determine the features for the output. The network can be, in some embodiments, either a standard pre-trained network where weights have been previously determined (e.g., optimized) and loaded onto the network, or alternatively, the network can be initially a standard network, but is then trained to improve specific recipient results based on outcome oriented reinforcement learning techniques.

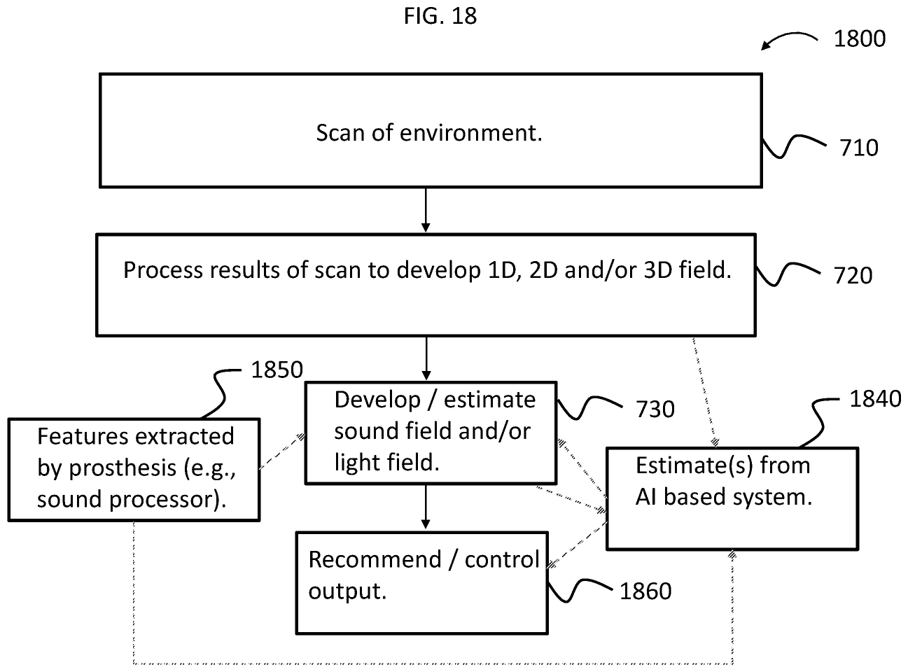

FIG. 18 presents an exemplary algorithm for an exemplary method, method 1800, that includes many of the actions, in fact all the actions, of method 700 detailed above. The like method actions described above will not be addressed here, but as can be seen, there are the addition of method actions 1840 and 1850. Method action 1850 includes utilizing the prostheses (or other component of a system of which the prosthesis is a part), to extract certain features associated with the ambient environment, typically with respect to embodiments that utilize a hearing prosthesis, a sound environment. These features can be utilized in method action 730 to develop estimate the sound field and/or light field. This can be done without utilizing artificial intelligence/trained neural network technology, but as can be seen, in an exemplary embodiment, the results of method action 850 can be provided to an AI based system, as represented by the dashed arrow extending from block 1850 to block 1840. And in this regard, method action 1840 includes obtaining estimates from an AI based system, such as the trained neural network and/or a DNN and/or the results of such, etc. As can be seen, method action 1840 can be executed utilizing the results of method action 720 and/or the result of method action 1850. This is detailed above, but briefly, in at least some embodiments, the features of the sound environment can be utilized to inform or at least validate the understanding of the structural environment in which the prostheses and/or recipient or otherwise the sensors are located or were located. In any event, as seen, the results of the estimate from the AI based system can be provided otherwise utilized with method action 730 to develop the sound field and/or the light field. And to be clear, method action 730 can be executed as part of an artificial intelligence system or otherwise utilizing the machine learning features of the results thereof detailed herein (as represented by the two arrows between block 730 and block 1840). Moreover, as seen, the estimates from the artificial intelligence-based system can be utilized to recommend and/or control the output (to control the prosthesis or to provide a recommendation to the recipient, etc.).

And note that of a given LiDAR data set to implement the teachings detailed herein. In some embodiments, statistical databases can be accessed to correlate a given structural arrangement or spatial arrangement with certain settings and/or certain properties that have utilitarian value with respect to improving the utilization of the prostheses are otherwise the efficacy of the proceedings. Embodiments thus include continuously or semi-continuously or otherwise periodically collating and collecting data across multiple users and utilizing this data to train the neural networks are otherwise improve the training thereof. Accordingly, embodiments include data collection techniques with respect to any utilitarian data that can enable machine learning to improve or otherwise be implemented to affect at least some of the teachings detailed herein.

It is noted that any method action disclosed herein corresponds to a disclosure of a non-transitory computer readable medium that has program there on a code for executing such method action providing that the art enables such. Still further, any method action disclosed herein where the art enables such corresponds to a disclosure of a code from a machine learning algorithm and/or a code of a machine learning algorithm for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code-code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the utilitarian value of any one or more of the implants detailed herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A system, comprising:
a light sensor and/or a sonic sensor and/or a radio wave sensor; and
a processor configured to receive input based on light and/or sonic frequency reflection and/or radio wave reflection captured by the respective sensor and analyze the received input to:
develop a data usable to control a sensory device based on the received input.

2. The system of claim 1, wherein:
the system includes the device, and the developed data controls an aspect of the prosthesis.

3. The system of claim 2, wherein:
the device is a hearing prosthesis.

4. The system of claim 1, wherein:
the system includes the light sensor and the input is based on light captured by the light sensor.

5. The system of claim 1, wherein:
the light sensor is part of a LiDAR sub-system that is part of the system;
the processor is configured to develop beamforming data based on the received input; and
the processor is configured to develop the data to control the device based on beamforming data to implement a beamforming algorithm.

6. The system of claim 1, wherein:
the processor is configured to develop second data indicative of a two-dimensional and/or a three-dimensional digital model of the area and/or volume, respectively, proximate the respective sensor; and
the processor is configured to develop the data usable to control the device based on the second data.

7. (canceled)

8. The system of claim 1, wherein:
the system is configured to perform an alarm function to warn a recipient of the sensory device based on object detection based on the received input.

9. The system of claim 1, wherein:
the developed data is a control signal; and
the system includes the sensory device, circuitry of the sensory device being in signal communication with a component of the system that generates the control signal.

10. The system of claim 1, wherein:
the sensory device is a vestibular balance prosthesis, wherein the prosthesis includes LiDAR capable light capture devices.

11. A non-transitory computer readable medium having recorded thereon, a computer program for executing at least a portion of a method, the computer program including:
code for analyzing first data based on data captured by a light sensor and/or sonic sensor and/or radio wave sensor; and
code for quantitatively and/or qualitatively identifying a device performance impacting feature of an environment in which the light sensor and/or sonic sensor and/or radio wave sensor is located based on the analysis of the first data.

12. The medium of claim 11, wherein:
the feature is a distance to an object in the environment from the light sensor and/or the sonic sensor and/or the radio wave sensor and/or a sensor of device or another sensor of a system of which the device is a part.

13. The medium of claim 11, wherein:
the feature is an angle to an object in the environment from the light sensor and/or the sonic sensor and/or the radio wave sensor and/or a sensor of the device or another sensor of a system of which the device is a part.

14. The medium of claim 11, wherein:
the device is a sensory prosthesis, and the device impacting feature is a feature that is accommodated by a real-time adjustment to the sensory prosthesis during use of the sensory prosthesis.

15. The medium of claim 11, further comprising:
code for automatically controlling the device/sensory prosthesis based on the identified feature.

16-17. (canceled)

18. The medium of claim 11, further comprising:
code for determining a 3D field in which the respective sensor is located, wherein the code for identifying the feature uses the determined 3D field to identify the feature.

19. (canceled)

20. The medium of claim 11, wherein:
the computer program is a dual purpose hearing habilitation and/or rehabilitation program and a real time hearing perception improvement program, wherein the device is a sensory prosthesis.

21. A method, comprising:
obtaining first data based on captured light and/or sonic energy and/or radio frequency energy captured during a first temporal period utilizing an electronic device during the first temporal period; and
at least one of:
identifying an action to improve efficacy of a prosthesis based on the obtained first data; or
developing a sensory habilitation and/or rehabilitation tool and/or engaging in a sensory habilitation and/or rehabilitation program based on the obtained first data.

22. The method of claim 21, further comprising:
identifying an action to improve efficacy of a prosthesis based on the obtained first data, wherein the action improves perception of captured sound and/or light by a recipient of a sensory prosthesis based on the obtained first data.

23. (canceled)

24. The method of claim 22, further comprising:
capturing sound and/or light during the first temporal period and/or during a second temporal period subsequent the first temporal period but where the first temporal period and the second temporal period are sufficiently close that the first and second temporal periods can be considered real time or near real time, utilizing at least one hearing prosthesis, wherein the captured sound and/or light is captured in a manner at least partially based on the identified action.

25. The method of claim 22, further comprising:
capturing sound and/or light during the first temporal period and/or during a second temporal period subsequent the first temporal period utilizing at least one hearing prosthesis; and
evoking a sensory percept based on the captured sound and/or light at least partially based on the identified action.

26. The method of claim 21, further comprising:
developing the habilitation and/or rehabilitation tool based on the obtained first data.

27. The method of claim 21, further comprising:
developing the habilitation and/or rehabilitation tool based on the obtained first data, wherein the tool includes a virtual reality component using images developed from the obtained first data.

28-32. (canceled)
